(19) United States
(12) Patent Application Publication
(10) Pub. No.: US 2012/0276530 A1
(43) Pub. Date: Nov. 1, 2012

(54) LABEL-FREE SENSING OF PNA-DNA COMPLEXES USING NANOPORES

(75) Inventors: Amit Meller, Brookline, MA (US); Maxim Frank-Kamenetskii, Brookline, MA (US); Meni Wanunu, Chestnut Hill, MA (US); Heiko Kuhn, Seeheim-Jugenheim (DE); Alon Singer, Brighton, MA (US); Will Morrison, Allston, MA (US)

(73) Assignee: TRUSTEES OF BOSTON UNIVERSITY, Boston, MA (US)

(21) Appl. No.: 13/391,695

(22) PCT Filed: Aug. 24, 2010
(86) PCT No.: PCT/US10/46403

§ 371 Date: Jul. 17, 2012

Related U.S. Application Data
(60) Provisional application No. 61/236,187, filed on Aug. 24, 2009.

(51) Int. Cl. G01N 27/26; B82Y 5/00

(52) U.S. Cl. 435/6.11; 436/501; 435/5; 435/6.15; 977/781; 977/920

(57) ABSTRACT

Embodiments disclosed herein relate to a method of detecting specific DNA sequences and the application of this method in the detection of pathogens, viruses, drug-resistant pathogens, genomic variations associated with disease/disorder susceptibility etc. based on specific signature sequences unique to the pathogens, viruses, drug-resistant pathogens or genomic variations. The method can also be used to distinguish a pool of same-sized dsDNA on the basis of sequence differences. The method uses non-optically labeled bis-PNA and/or gamma-PNA probes to tag specific target sequences for identification by solid-state nanopores.

### CROSS REFERENCE TO RELATED APPLICATION

This application claims benefit under 35 U.S.C. §119(e) of the U.S. Provisional Application No. 61/236,187 filed Aug. 24, 2009, the contents of which are incorporated herein by reference in its entirety.

### GOVERNMENT SUPPORT

This invention was made with Government support under contract No. HG-004128 awarded by the National Institute of Health and contract No. PHY-0646637 awarded by the National Science Foundation. The Government has certain rights in the invention.

### BACKGROUND OF INVENTION

The ability of nucleic acids to spontaneously form stable, sequence-specific complexes with other nucleic acids, which serve as molecular probes, has been exploited for a wide range of applications in life sciences, biotechnology, medicine, and forensics. Examples range from polymerase chain reaction (PCR) and fluorescence in situ hybridization (FISH) to DNA microarrays and sequencing by hybridization. Current methods for detection of nucleic acids of interest employ such sequence-specific probes that are labeled in various ways to facilitate visualization and detection of the nucleic acids of interest. For example, in Southern blots, the probes are labeled with radioisotopes such as ³²P and ¹²⁵I.

However, one factor limiting the adaptability of this process to a greater number of applications is the large negative linear charge density inherent in nucleic acid strands, which significantly reduces the stability of nucleic acid complexes. In the various applications, e.g. PCR, FISH, Southern blots etc, the conditions of hybridization and washing have to be carefully controlled for sufficient stability of the hybridized complex to ensure sensitivity, specificity and accuracy of the results from the probes.

Innovations that improve the stability of nucleic acid complexes can lead to more sensitive, specific and accurate methods for detection of nucleic acids of interest.

Various DNA detection systems that use nanopores have been proposed, each with their respective limitations in application in the field. For example, U.S. Pat. No. 6,015,714 proposed a method for sequencing DNA by distinguishing bases of DNA using the highly sensitive signals of nanopores, the method includes providing a small pore between two otherwise not connected pools or reservoirs, the pore connects the two pools. DNA biopolymer can be placed in one pool, and measurements are taken as the biopolymer passes through the pore. However, current literature indicates that nanopore sequencing is still at the proof-of-concept experimental stage, with some laboratory-based data to back up the different components of the sequencing method, but not yet parallelized, routineized, nor cost-effective enough to compete with other “next generation sequencing” methods. In particular, a resolution that is able to detect single bases remains to be achieved.

U.S. Pat. No. 6,362,002 discloses a method of distinguishing a single-stranded nucleic acid from a double-stranded (ds) nucleic acid by providing a nanopore allowing sequential passage of bases of a single-stranded DNA. In this disclosure, a ds nucleic acid passes through a nanopore at a rate slower than that of a single-stranded (ss) nucleic acid, because the ds nucleic acid may be separated into single-stranded nucleic acids during its passage through the nanopore. The method does not facilitate distinguishing a pool of same-sized ss nucleic acid and/or ds nucleic acids on the basis of sequence differences. It is not uncommon to encounter mixtures of different nucleic acids having the same size (by length) but are otherwise different sequences out in the field.

U.S. Patent Publication No. 2003/0104428 proposed a prophetic method for characterizing a DNA sample using a nanopore based on the determination of a specific sequence using either a substance recognizing a specified local area in a protein or DNA and observing changes in the signal amplitude caused by other substances that are bound to the DNA, thus detecting the specific base sequence of the DNA. Specifics of the recognition criteria in a protein or DNA and the stability of the recognition during detection were not well defined. Binding substances such as peptide nucleic acids (PNA) that can form very stable PNA-DNA complexes are not mentioned. The complex formed by substances bound on to DNA need to be sufficiently stable in order to generate a stable signal and for the stable signal to be detected, especially in a longer polymer>1 kilobases (kb). Therefore, the recognition of a specified local area on the DNA and stability of the complex are likely to play a major role to practicing this method. In the disclosure, polymers of <1 kb were proposed and no actual working example was provided.

U.S. Pat. No. 6,428,959 discloses a method of distinguishing a ss nucleic acid from a ds nucleic acid. The method includes translocating nucleic acids in an aqueous sample through a nanopore having a diameter ranging from 3 to 6 nanometers (nm) and monitoring the current amplitude through the nanopore during the translocating process. The size of the ss or ds DNA is limited to ˜100 mer and it does not facilitate actual detection of specific sequence of interest within the DNA.

These prior-art DNA detection methods that use nanopores raise problems, because when these methods are applied, the detection of DNA becomes difficult when the size is larger than 1000 base pairs (bp). Moreover, it becomes more difficult when the detection is sequence specific and these specific sequences are dispersed over 100s of by apart on a single DNA, and the sequence are small (<8 bp) due to the very small contrast in signal amplitude or electric current differential produced and the difficulty in detecting the small current differential in the nanopore over the background noise.

### SUMMARY OF THE INVENTION

Disclosed herein, the inventors were able to demonstrate for the first time the electrical detection of individual specific sequences in dsDNA of >1 kb on the basis of PNA stably binding to specific sequences that are spaced at 850 bp apart. For example, the size of the dsDNA is 3.5 kb. The electrical detection of non-optically labeled dsDNA-PNA complexes is at sub-nM solution concentrations. The bis-PNA invaded target sequences can be easily identified in a DNA fragment solely by the ion-current signatures of the threaded molecules. The method comprises threading of dsDNA duplexes tagged with sequence-specific bis-PNA probes through solid-state nanopores, while monitoring the ion current of an electrolyte in the solution through the same nanopore. With this method, the inventors also show that it is possible to distinguish a pool of same-sized dsDNA on the basis of sequence differences.

The inventors have successfully shown that it is possible to detect <8 bp DNA sequences on large double stranded DNA (dsDNA) (>1 kb) solely using an electrical, label free detection method with a solid-state nanopore apparatus. Moreover, the inventors showed that single molecules of DNA can be detected with the method. Specific sequences on large dsDNA were tagged with peptide nucleic acids (PNA) that are designed to complement base pair with the desired specific sequences on the dsDNA. The dsDNA-PNA complex thus formed is identified as distinct decreases/differential in electric current flowing through a nanopore Unlike other nucleic acid hybridization complexes, the contrast between this dsDNA-PNA complex and the non-PNA complexed DNA is sufficiently large enough to produce a distinct detectable and readable electric current differential in the nanopore detection apparatus. The inventors also show that several PNAs can be used to tag a single dsDNA and the positions of the dsDNA-PNAs complexes correlates with the distinct decreases in electric current flowing through a nanopore over time. The patterns created by the distinct decreases in electric current when a particular set of probes are used form a unique identification code for the dsDNA.

In addition, the inventors were also able to discriminate between dsDNA that have different sequences but identical lengths using one or more PNAs. The discrimination is by way of the sequence-specific PNA which hybridizes to the dsDNA.

Accordingly, embodiments of the invention provides methods for detecting a ds biomolecule of interest comprising selecting at least one probe having a known sequence that hybridize by complementary base pairing to a specific region on a ds biomolecule and contacting the at least one probe with the ds biomolecule such that the probe attaches to the specific region of the ds biomolecule to produce a probe-biomolecule complex, wherein the complex has sufficiently large cross-sectional surface area that produces a contrast in a signal amplitude that is detectable, for example, by producing a distinct detectable and readable electric current differential in a nanopore detection apparatus.

In one embodiment, the method of detecting a ds biomolecule of interest comprises the steps of: providing a sample comprising a ds biomolecule; providing at least one probe having a known sequence; contacting the at least one probe with the ds biomolecule such that the probe attaches to a specific region of the ds biomolecule to produce a probe-biomolecule complex; introducing the probe-biomolecule complex into a microfluidic solid-state nanopore detection apparatus comprising a first fluid chamber, a second fluid chamber, a nanopore positioned between the first and second chambers such that the first and second chambers are in fluid communication via the nanopore; translocating the probe-biomolecule complex from the first chamber through the nanopore and into the second chamber by applying an electric potential between the two chambers; monitoring changes in current across the nanopore as the probe-biomolecule complex is translocated therethrough, the change in electrical current corresponding to presence of the probe-biomolecule complex containing the probe; and recording the changes in electrical current as a function of time.

In one embodiment, the method of distinguishing biomolecules having the same length or size or charge is provided, the method comprising providing a sample comprising at least two ds biomolecules of the same length; providing at least one probe having a known sequence; contacting the at least one probe with the ds biomolecules such that the probe attaches to a specific region of the ds biomolecule to produce a probe-biomolecule complex; introducing the sample and probe into a microfluidic solid-state nanopore detection apparatus comprising a first fluid chamber, a second fluid chamber, a nanopore positioned between the first and second chambers such that the first and second chambers are in fluid communication via the nanopore; translocating the sample and probe from the first chamber through the nanopore and into the second chamber by applying an electric potential between the two chambers; monitoring changes in current across the nanopore as the sample and probe is translocated therethrough, recording the changes in electrical current as a function of time, wherein a change in electrical current corresponding to presence of the probe-biomolecule complex containing the probe and indicating the presence of at least one ds biomolecule having specific region for the probe, and wherein no change in electrical current corresponding to absence of the probe-biomolecule complex containing the probe and indicating the presence of at least one ds biomolecule that does not have specific region for the probe.

Embodiments of the invention also provides a method of diagnosing a drug resistant strain of pathogenic bacteria in a specimen under DNA non-denaturing conditions, the method comprising: providing a sample containing DNA; providing at least one probe having a known sequence that is unique to a drug resistant strain of pathogenic bacteria; contacting the at least one probe with the sample to produce a probe-DNA complex; introducing the probe-DNA complex into a microfluidic solid-state nanopore detection apparatus comprising a first fluid chamber, a second fluid chamber, a nanopore positioned between the first and second chambers such that the first and second chambers are in fluid communication via the nanopore; translocating the probe-DNA complex from the first chamber through the nanopore and into the second chamber by applying an electric potential between the two chambers; monitoring changes in current across the nanopore as the probe-DNA complex is translocated therethrough and recording the changes in electrical potential as a function of time, wherein the change in electrical potential corresponding to presence of the probe-DNA complex containing the probe, indicating the presence of the drug resistant strain of pathogenic bacteria in the sample.

In another embodiment, the invention provides a method of detecting a drug resistant strain of Staphylococcus aureus in a specimen under DNA non-denaturing conditions, the method comprising: providing a sample containing DNA; providing at least one probe having a known sequence that is unique to a drug resistant strain of Staphylococcus aureus; contacting the at least one probe with the sample to produce a probe-DNA complex; introducing the probe-DNA complex into a microfluidic solid-state nanopore detection apparatus comprising a first fluid chamber, a second fluid chamber, a nanopore positioned between the first and second chambers such that the first and second chambers are in fluid communication via the nanopore; translocating the probe-DNA complex from the first chamber through the nanopore and into the second chamber by applying an electric potential between the two chambers; monitoring changes in current across the nanopore as the probe-DNA complex is translocated therethrough and recording the changes in electrical current as a function of time, wherein the change in electrical current corresponding to presence of the probe-DNA complex containing the probe, indicating the presence of the drug resistant strain of Staphylococcus aureus in the sample.

In some aspect, the invention also provides a method of diagnosing the presence of a pathogenic bacteria or virus in a specimen under DNA non-denaturing conditions, the method comprising: providing a sample containing DNA; providing at least one probe having a known sequence that is unique to a pathogenic bacteria or virus; contacting the at least one probe with the sample to produce a probe-DNA complex; introducing the probe-DNA complex into a microfluidic solid-state nanopore detection apparatus comprising a first fluid chamber, a second fluid chamber, a nanopore positioned between the first and second chambers such that the first and second chambers are in fluid communication via the nanopore; translocating the probe-DNA complex from the first chamber through the nanopore and into the second chamber by applying an electric potential between the two chambers; monitoring changes in current across the nanopore as the probe-DNA complex is translocated therethrough and recording the changes in electrical current as a function of time, wherein the change in electrical current corresponding to presence of the probe-DNA complex containing the probe, and indicating the presence of the pathogenic bacteria or virus in the sample.

Similarly, in some embodiments, the invention provides methods for detecting mutations in the sequences, e.g. single nucleotide polymorphisms, repeat nucleotides etc. Some of these mutations are known biomarkers for risk factors in developing certain diseases such as cancer, familial early onset Alzheimer's disease and/or susceptibility to drug reaction or response.

In one embodiment, the ds biomolecule is a ds DNA. In one embodiment, the ds biomolecule is a RNA/DNA hybrid. In one embodiment, the at least one probe is a PNA. Other probes include RNA, DNA, and modified forms thereof. In another embodiment, the PNA is a bis-PNA. In another embodiment, the PNA is a gamma-PNA (γ-PNA). In one preferred embodiment, the γ-PNA can have a higher binding to affinity to DNA. For example, the γ-PNA has a modified nucleobase, guanidinium G-clamp (X) that replaces cytosine in the canonical G:C binding. The G-clamp results in increased thermal stability of matched duplexes due to formation of five hydrogen bonds with guanine. The probe's function is to hybridize to the ds biomolecule by complement base pairing to form a stable complex. Not the entire probe needs to hybridize to the ds biomolecules. In one embodiment, at least 50% of the probe hybridizes to the ds biomolecule. In another embodiment, at least 20% of the probe hybridizes to the ds biomolecule. In other embodiments, at least 5%, at least 10%, at least 15%, at least 25%, at least 30%, at least 35%, at least 40% or at least 45% of the probe hybridizes to the ds biomolecule. In some embodiments, the probe is a hybrid of PNA, RNA, or DNA. In some embodiments, the hybridization portion of the probe is a hybrid of PNA, RNA, or DNA. In some embodiments, at least 50% of the hybridization portion of the probe is a PNA. Modifications to the probes can be included to further increase the size/cross-sectional surface area of the probe-ds biomolecules thus formed. This serves to increase electric current differential for detection purposes.

In one embodiment, when at least two probes are used, the probes attach to different specific regions of the ds biomolecule or dsDNA and the probe-binding regions on the ds biomolecule or dsDNA are at least 50 bp apart.

In one embodiment, the probe-biomolecule complex is a triplex, i.e. comprising three strands of nucleic acid. In another embodiment, the PNA-dsDNA complex is a triplex. In yet another embodiment, the bis-PNA-DNA complex is a triplex.

In one embodiment, the nanopore in the solid-state detection apparatus is between 3-6 nm in diameter. In another embodiment, the nanopore is up to 10 nm in size. In another embodiment, the electric potential nanopore detection apparatus is between 50-1000 mV.

In one embodiment, the specimen is a mixture of bacteria cells and non-bacteria cells. A sample comprising a mixture of dsDNA is derived from this specimen.

In one embodiment, the specimen is a mixture of different types of bacteria. A sample comprising a mixture of dsDNA is derived from this specimen.

In one embodiment, the specimen is obtained from the group consisting of: blood, sputum, feces, saliva, peritoneal fluid, synovial fluid, urine, body tissue, cerebrospinal fluid, soil, water, rain, sewage, air, food, dust, and solid surface wipes.

In one embodiment, the pathogenic bacteria is selected from the group consisting of Clostridium botulism, Clostridium difficile, Bordetella pertussis, Listeria monocytogenes, Neisseria meningitides, Haemophilus influenzae, Brucella species, Coxiella burnetii, Shigella species, Escherichia coli O157:H7, Mycoplasma pneumoniae, Mycoplasma tuberculosis, Mycoplasma avium-intracellular complex, Mycoplasma gordonae, Mycoplasma kansaii, Staphylococci aurenus, Staphylococci epidermidis, Staphylococci saprophiticus, Staphylococci lugdunensis, Streptococcus pyogenes, Streptococcus agalactiae, Streptococcus pneumoniae Enterococcus faecalis, Enterococcus faecium, Enterococcus gallinarum, Enterococcus casseliflavus, Klebsiella pneumoniae, Klebsiella oxytoca, Pseudomonas aeruginosa, Acinetobacter baumannii, Nocardia species, Salmonella species, Vibrio species, and Yersinia.

In one embodiment, the drug resistant strain of Staphylococcus aureus is resistant to a group of drugs consisting of methicillin, clindamycin, ciprofloxacin and vancomycin.

In one embodiment, the drug resistant strain pathogenic bacteria is resistant to a group of drugs consisting of methicillin, macrolide, lincosamide, streptogamin, and vancomycin.

In one embodiment, the drug resistant strain pathogenic bacteria is selected from a group consisting of Staphylococcus, Steptococcus, Mycoplasma, Pneumococcus, Acinetobacter and Entercoccous.

### BRIEF DESCRIPTION OF THE DRAWINGS

FIG. 1a shows a schematic illustration of a double-stranded DNA (dsDNA) molecule with two bis-PNA probes threaded through a 4 nm SiN pore. Voltage bias is used to facilitate the translocation of a DNA molecule from cis to trans.

FIG. 1b shows the schematics of the 3,500 base-pair (bp) dsDNA PCR fragments used in the example. F1 is a control molecule having no binding sites for the bis-PNA. F2 contains two binding sites separated by 855 bp.

FIG. 1c shows the hybridization of the two bis-PNA probes with the dsDNA to form a DNA-PNA triplex at the point of probe binding.

FIG. 1d shows the gel-shift analysis of the DNA-PNA complexes: F1 (lane 1), F2 with one of the two PNA probes (lane 2 and 3), F2 with both PNA probes (lane 4) and a dsDNA marker (M).

FIG. 2a shows representative ion current traces of F1 translocation through a ˜4.5 nm pore, after incubation with the two PNA probes (P1 and P2).

FIG. 2b shows representative ion current traces of F2 translocation through a ˜4.5 nm pore, after incubation with the two PNA probes (P1 and P2).

FIG. 3 shows a scatter plot describing the change in the mean ion current versus its duration of each translocation event of F 1P1P2 (dark grey) and F2P1P2 (lighter grey) (>1,000 DNA translocation events shown per molecule), measured using the same ˜4.5 nm pore.

FIG. 4 shows a hypothetical signature site and the hybridization of two different bis-PNAs to the signature site to form a triplex invasion structure comprising a loop called a P-loop.

FIG. 5 shows a schematic illustration of the PNA/DNA complexes used for the preliminary studies described herein. The target sequence forms a triplex invasion structure with the bis-PNA tags/probes, while the complementary strand forms a loop called a P-loop.

FIGS. 6a and 6b show the detection of PNA/DNA complexes using nanopores.

FIG. 6a is a display of five typical events of a control molecule (DNA, 2,700 bp) with not attached bis-PNA probe. The corresponding histogram displays two levels (open pore and the DNA level).

FIG. 6b is a display of five typical events of DNA/PNA complexes. These events display an additional current level attributed to the DNA/PNA complexes.

FIG. 7a is an exemplary bis-PNA oligomer, similar to those used in the project. Two homopyrimidine PNA oligomers are connected by a flexible linker and flanked by three lysine residues, which are positively charged at neutral pH; in one of the two oligomers all Cs are replaced by the J base shown in FIG. 7c.

FIG. 7b are bis-PNAs carrying normal bases are capable of binding to dsDNA forming the P-loop (FIG. 4) in which a triplex with the purine strand is assembled consisting of canonical TAT and CGC+ base triades (the latter is shown); since in the CGC+ base triade the C forming Hoogsteen pair must be protonated, the binding of normal-base bis-PNA is strongly pH-dependent.

FIG. 7c are bis-PNAs in which on one of oligomers all Cs are replaced with pseudoisocytosines (the J base).

FIG. 8a shows the chemical structure of a chiral γ-PNA monomer which is structurally different in its unbound form, i.e. before binding to the DNA target site. The letter B in bold indicates the position of a nucleobase (either A, T, C, G, X or other synthetic nucleobases). After binding the γ-PNA is identical to essentially most other single stranded PNA forms, unlike the bis-PNA which contains a markedly different bound structure.

FIG. 8b shows of the interactions between a guanosine with a synthetic cytosine nucleobase labeled X, G:X. This G:X interaction has enhanced affinity compared to the canonical G:C interaction due to the five hydrogen bonds in the G:X interaction compared to only three hydrogen bonds in the G:C interaction.

FIG. 8c shows an exemplary sequence of a γ-PNA (SEQ. ID. NO: 86).

FIG. 8d shows a cartoon of when γ-PNA binds to DNA. The new structure is a duplex invasion and not a triplex invasion, as in bis-PNA.

FIG. 9a shows representative ion current traces of a control dsDNA (1000 bp) without any bound γ-PNA translocating through a ˜3.5 nm pore.

FIG. 9b shows representative ion current traces of a dsDNA (1000 bp) with a γ-PNA bound thereon at the mid point (500 bp) translocating through a ˜3.5 nm pore.

### DETAILED DESCRIPTION OF THE INVENTION

The inventors have improved the stability and increased the size/cross-sectional surface area of nucleic acid complexes comprising probes by using a synthetic form of nucleic acids, peptide nucleic acids (PNA), as the molecular probe Unlike a deoxyribonucleic acid (DNA) or ribonucleic acid (RNA) probe, PNA lacks a net electrical charge along its protein-like backbone and therefore do not contribute to the large negative linear charge density in the complex thus formed. This in turn increased the stability of nucleic acid complexes. Moreover, the unique design of a particular type of PNA, in particular, a bis-PNA, greatly increases the size/cross-sectional surface area of complexes thus formed, and in turn aids in their detection, for example, by an electrical nanopore detection strategy.

In addition, the inventors utilized a micro-fluidic solid state nanopore technique to detect the unlabeled dsDNA-PNA complexes. The inventors were able to demonstrate for the first time the electrical detection of individual unlabeled dsDNA-PNA complexes at sub-nM solution concentrations and that the bis-PNA-invaded target sequences can be easily identified in a DNA fragment solely by the ion-current signatures of the threaded molecules. The method comprises threading of dsDNA duplexes tagged with sequence-specific bis-PNA probes through solid-state nanopores, while monitoring the ion current of an electrolyte in the solution through the same nanopore.

The method is applicable for detecting individual double-stranded DNA molecules (dsDNA) having specific sequences of interest, dsDNA that are >1 kb long. The method can be used for detecting multiple specific sequences of interest on an individual dsDNA. The method does not involve DNA amplification, the use of any enzymatic reaction or any form of labeling in order to visualize the dsDNA. Instead, the method uses peptide nucleic acid oligomers (PNA) to ‘tag’ the specific sequences of interest on the dsDNA.

The PNA is not optically labeled, meaning, the PNA is not labeled such that it can be detected optically, e.g. by fluorescence or visible color or radioactive decay. PNA are synthetic nucleic acid analogs that mimic but have a pseudopeptide backbone instead of a phosphate-sugar backbone. As such, PNA can hybridize to form double-stranded structures with DNA in a similar fashion as naturally occurring nucleic acids. The dsDNA-PNA complexes thus formed are ‘detected’ as changes in an electric current through a nanopore.

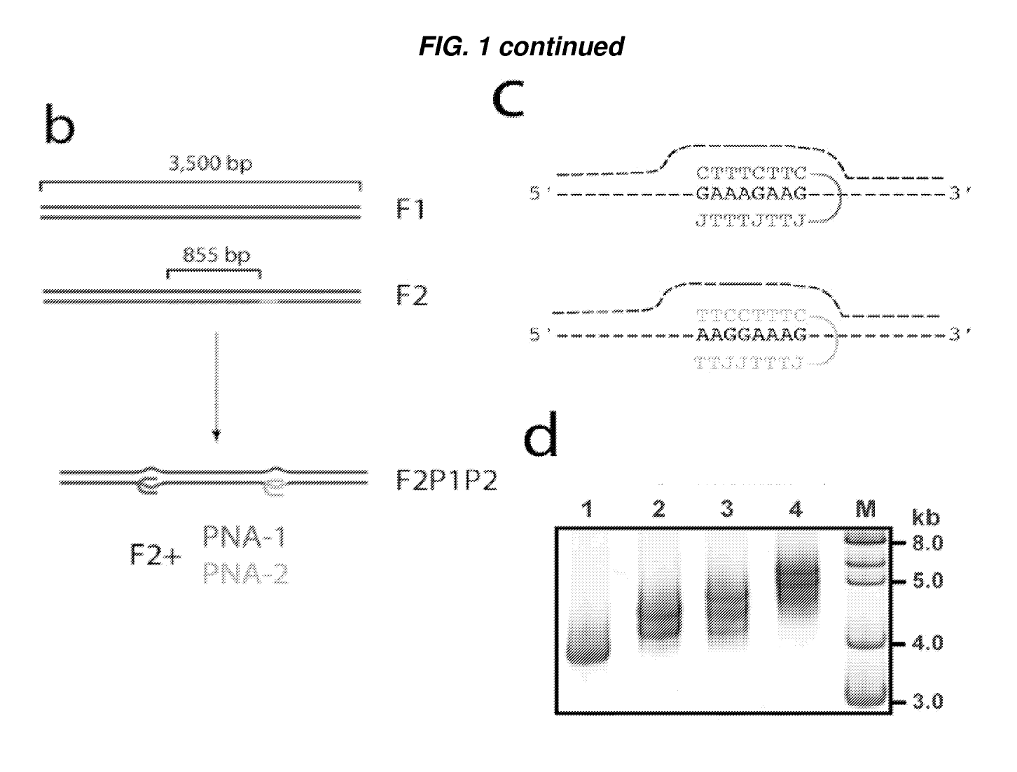

Individual dsDNA tagged with sequence-specific PNA(s) is placed in an electric field within a solid-state nanopore apparatus comprising a first fluid chamber, a second fluid chamber, and a nanopore positioned between the first and second chambers such that the first and second chambers are in fluid communication via the nanopore. The electric current flowing through the nanopore is monitored. By virtue of the negative charge of the dsDNA, the dsDNA is forced to translocate from one chamber to the other by passing through the nanopore. When the dsDNA enters the nanopore and begins to translocate across, the pore becomes partially blocked by the dsDNA, causing a drop in the electric current flowing through the pore. When the region on the dsDNA with the specific sequence of interest reaches and enters the pore, the specific sequence of interest that is now complexed with a complementary base paired PNA, the pore is blocked further, causing an additional drop in the electric current. By monitoring the changes in electric current over time as the whole dsDNA molecule translocte through the pore, it is possible to detect single dsDNA molecule with specific sequence(s) of interest. Multiple specific sequence(s) of interest registered as multiple drops in electric current over time (FIG. 2).

### DEFINITIONS OF TERMS

As used herein, the term “peptide-nucleic acid” or “PNA” refers to any synthetic nucleic acid analog (deoxyribonucleic acid (DNA) mimics with a pseudopeptide backbone) which can hybridize to form double-stranded structures with DNA in a similar fashion as naturally occurring nucleic acids. PNA is an extremely good structural mimic of DNA (or of ribonucleic acid (RNA)), and PNA oligomers are able to form very stable duplex structures with Watson-Crick complementary DNA and RNA (or PNA) oligomers, and they can also bind to targets in duplex DNA by helix invasion. Other type of complementary base pairing, such as the Hoogsteen pairing is possible too. PNA can be an oligomer, linked polymer or chimeric oligomer. Methods for the chemical synthesis and assembly of PNAs are well known in the art and are described in U.S. Pat. Nos. 5,539,082, 5,527,675, 5,623,049, 5,714,331, 5,736,336, 5,773,571, and 5,786,571. Uses of the PNA technology are also well known in the art, see U.S. Pat. Nos. 6,265,166, 6,596,486, and 6,949,343. These references are hereby incorporated by reference in their entirety.

Modification can be included in the pseudopeptide backbone to change the overall charge of the PNA, for example, selection of more charged amino acids instead of non-polar amino acids serves to increase the charge of the PNA oligomer. In addition, small particle, molecules, protein, or peptides can be conjugated to the pseudopeptide backbone to enhance the bulk of the dsDNA-PNA complex. Enhance bulk serves to enhance the signal amplitude so that any electrical current differential resulting from the increase in bulk can be easily detected. Examples of small particle, molecules, protein, or peptides that can be conjugated to the pseudopeptide backbone include but are not limited to nanometer-sized gold particles (e.g. 3 nm), quantum dots, polyethylene glycol (PEG), polyvinyl pyrrolidone, polyvinyl alcohol, polyamino acids, divinylether maleic anhydride, N-(2-Hydroxypropyl)-methacrylamide, dextran, dextran derivatives including dextran sulfate, polypropylene glycol, polyoxyethylated polyol, heparin, heparin fragments, polysaccharides, cellulose and trypsin inhibitor. Method of conjugation of molecules are well know in the art, e.g. in U.S. Pat. Nos. 5,180,816, 6,423,685, 6,706,252, 6,884,780, and 7,022,673, which are hereby incorporated by reference in their entirety. Examples of some conjugating agents include but are not limited to ethylenediaminetetraacetic acid (EDTA), diethylenetriaminopentaacetic acid (DTPA), ethyleneglycol-0,0′-bis(2-aminoethyl)-N,N,N′,N′-tetraacetic acid (EGTA), N,N′-bis(hydroxybenzyl)ethylenediamine-N,N′-diacetic acid (HBED), triethylenetetraminehexaacetic acid (TTHA), 1,4,7,10-tetra-azacyclododecane-N,N′,N″,N′″-tetraacetic acid (DOTA), 1,4,7,10-tetraazacyclotridecane-1,4,7,10-tetraacetic acid (TITRA), 1,4,8,11-tetraazacyclotetradecane-N,N′,N″,N′″-tetraacetic acid (TETA), and 1,4,8,11-tetraazacyclotetradecane (TETRA).

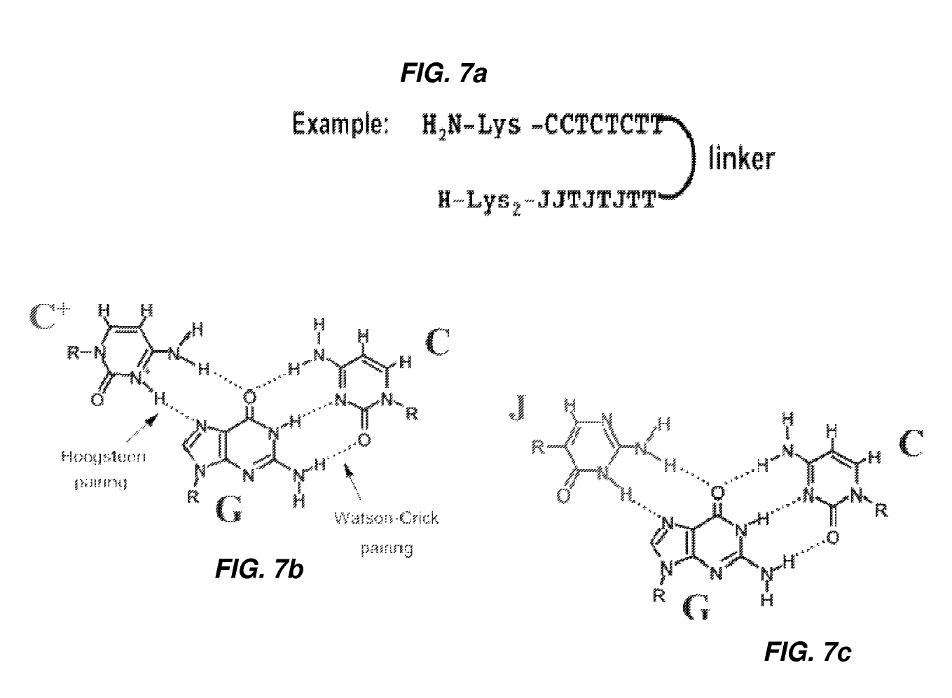

As used herein, the terms “bis-PNA” refers to two PNA oligomers connected by a flexible linker (see FIG. 7a). Bis-PNAs are the preferred PNA for invading and opening a duplex DNA strand to expose a single stranded DNA from the DNA duplex as bis-PNAs form stably DNA-PNA₂ triplexes with the duplex DNA (See WO96/02558). Bis-PNA molecules spontaneously invade dsDNA molecules with high affinity and sequence-specificity, owing to the simultaneous formation of Watson-Crick and Hoogsteen base-pairs. The designs and applications of PNA-openers are described in U.S. Pat. Nos. 6,265,166 and 6,596,486. The references disclosed herein are hereby incorporated by reference in their entirety.

As used herein, the term “comprising” means that other elements can also be present in addition to the defined elements presented. The use of “comprising” indicates inclusion rather than limitation.

As used herein, the term “complementary base pair” refers to A:T and G:C in DNA and A:U in RNA. Most DNA consists of sequences of nucleotide only four nitrogenous bases: base or base adenine (A), thymine (T), guanine (G), and cytosine (C) or pseudocytosine (J). The pairing is based on the Watson-Crick pairing or the Hoogsteen pairing. Together these bases form the genetic alphabet, and long ordered sequences of them contain, in coded form, much of the information present in genes. Most RNA also consists of sequences of only four bases. However, in RNA, thymine is replaced by uridine (U).

As used herein, the tern “non-denaturing conditions” refers to in the absence of high temperature>65° C. and/or strong base or acid that are pH<3 or >10, such as 1 M NaOH.

### Methods of the Invention

Accordingly, embodiments of the invention provides a method for detecting a ds biomolecule of interest, the method comprising selecting at least one probe having a known sequence that hybridizes by complementary base pairing to a specific region on a dsDNA biomolecules and contacting the at least one probe with the ds biomolecule such that the probe attaches to the specific region of the ds biomolecule to produce a probe-biomolecule complex, wherein the complex is sufficiently large cross-section surface area that produces a detectable contrast in signal amplitude or electric current change over that of a background, wherein the background in the signal amplitude or electric current corresponding to sections of non-PNA bound ds biomolecules, for example, by producing a distinct detectable and readable electric current differential in the nanopore detection apparatus.

One embodiment of the invention is a method for detecting a double-stranded or duplex (ds) biomolecule of interest, the method comprising the steps of: providing a sample comprising a ds biomolecule; providing at least one probe having a known sequence that hybridizes by complementary base pairing to a specific region on a dsDNA biomolecules; contacting the at least one probe with the ds biomolecule such that the probe attaches to a specific region of the ds biomolecule to produce a probe-biomolecule complex; introducing the probe-biomolecule complex into a microfluidic solid-state nanopore detection apparatus comprising a first fluid chamber, a second fluid chamber, a nanopore positioned between the first and second chambers such that the first and second chambers are in fluid communication via the nanopore; translocating the probe-biomolecule complex from the first chamber through the nanopore and into the second chamber by applying an electric potential between the two chambers; monitoring changes in current across the nanopore as the probe-biomolecule complex is translocated therethrough, wherein a change in electrical current corresponding to presence of the probe-biomolecule complex containing the probe, thereby indicating the presence of the ds biomolecule; and recording the changes in electrical current as a function of time.

Embodiments of the present invention also provides a method of distinguishing biomolecules having the same length or size or charge, the method comprising providing a sample comprising at least two ds biomolecules of the same length and also having sequence differences; providing at least one probe having a known sequence that hybridizes by complementary base pairing to a specific region on a dsDNA biomolecules; contacting the at least one probe with the ds biomolecules such that the probe attaches to a specific region of the ds biomolecule to produce a probe-biomolecule complex; introducing the sample and probe into a microfluidic solid-state nanopore detection apparatus comprising a first fluid chamber, a second fluid chamber, a nanopore positioned between the first and second chambers such that the first and second chambers are in fluid communication via the nanopore; translocating the sample and probe from the first chamber through the nanopore and into the second chamber by applying an electric potential between the two chambers; monitoring changes in current across the nanopore as the sample and probe is translocated therethrough, recording the changes in electrical current as a function of time, wherein a change in electrical potential corresponding to presence of the probe-biomolecule complex containing the probe and indicating the presence of at least one ds biomolecule having specific region for the probe, and wherein no change in electrical current corresponding to absence of the probe-biomolecule complex containing the probe and indicating the presence of at least one ds biomolecule that does not have specific region for the probe.

In one embodiment, the methods described herein of detecting a double-stranded or duplex (ds) biomolecule of interest is based on knowledge of the specific sequence inherent to the biomolecule of interest. The specific sequence inherent to the biomolecules of interest is used to design a probe that can hybridize to that specific sequence by complementary base pairing.

In one embodiment, the sample comprises a mixture of same sized ds biomolecules. The inventors have shown that the method facilitates distinguishing a mixture of same sized ds biomolecules with sequence differences on the basis of the specific sequences of the ds biomolecules.

In one embodiment, the probe has a known sequence. In one embodiment, the known sequence hybridizes to a specific sequence in a specific region of the ds biomolecules. In one embodiment, the hybridization is by complementary base pairing.

In one embodiment, the ds biomolecule is a dsDNA. In one embodiment, the dsDNA is at least 1 kb in length. In another embodiment, the dsDNA is 3.5 kb in length. In other embodiments, the dsDNA is at least 2 kb, at least 4 kb, at least 6 kb, at least 8 kb, at least 10 kb, at least 12 kb, at least 14 kb, at least 16 kb, at least 18 kb, at least 20 kb in length, including all the lengths between 1-20 kb.

In one embodiment, the at least one probe is a PNA. PNA is a synthetic form of nucleic acids which lacks a net electrical charge along its protein-like backbone. PNA has found a number of applications in vitro, and more recently in live cells to ‘tag’ specific sequences. In one embodiment, the at least one probe is a bis-PNA. A bis-PNA molecule is made of two PNA oligomers connected by a flexible linker A few lysine residues are often added at their termini to improve association kinetics to dsDNA. It can spontaneously invade dsDNA molecules with high affinity and sequence-specificity, owing to the simultaneous formation of Watson-Crick and Hoogsteen base-pairs. In other embodiments, the PNA can have certain modifications, such as those in (pseudocomplementary PNA (pcPNA) and gamma-PNA (γ-PNA). The synthesis of PNA are well known in the art and described in U.S. Pat. Nos. 5,539,082, 5,527,675, 5,623,049, 7,714,331, 5,736,336, 5,773571, and 5,786571. Uses of the PNA technology are also well known in the art, see U.S. Pat. Nos. 6,265,166, 6,596,486, and 6,949,343. These references are incorporated herein by reference in their entirety.

Generally, bis-PNAs comprise homopyrimidines or homopurines and its invasion of dsDNA generally requires a PNA2/DNA triplex formation. This essentially limits the target regions for hybridization on the dsDNA to homopurine homopyrimidine stretches. In order to avoid the sequence limitations associated with PNAs such as bis-PNAs so as to be able to target essentially any mixed DNA sequence, other modified PNA probes can used.

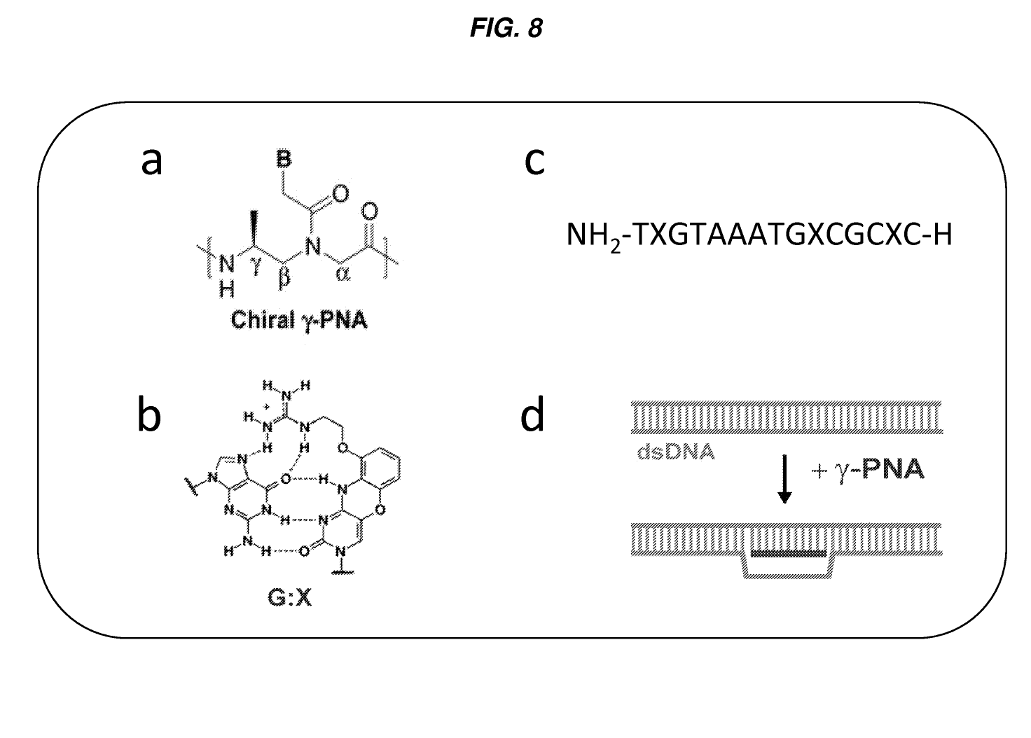

In another embodiment, the at least one probe is a γ-PNA. γ-PNA has a simple modification in a peptide-like backbone, specifically at the γ-position of the N-(2-aminoethyl)glycine backbone, thus generating a chiral center (See FIG. 8a) (Rapireddy S., et al., 2007. J. Am. Chem. Soc., 129:15596-600; He G, et al., 2009, J. Am. Chem. Soc., 131:12088-90; Chema V, et al., 2008, Chembiochem 9:2388-91; Dragulescu-Andrasi, A., et al., 2006, J. Am. Chem. Soc., 128:10258-10267). Unlike bis-PNA, γ-PNA can invade and bind to dsDNA without sequence limitation leaving one of the two DNA strands accessible for further hybridization. In addition, the inclusion of a modified nucleobase, guanidinium G-clamp (X) replacing cytosine in the γ-PNA produces in increased thermal stability of when the γ-PNA complement base pair to dsDNA due to the formation of five hydrogen bonds with guanine (See FIG. 8b). The modified “X”, when properly placed this can be used as a tool for increased sequence specificity.

The probe's function is to hybridize to the ds biomolecule by complement base pairing to form a stable complex and the complex that has sufficiently large cross-section surface area to produce a detectable change or contrast in signal amplitude over that of the background which are the signal amplitudes corresponding to sections of non-probe-bound ds biomolecules. The stability of the complex in important for the complex detection by the nanopore method. The complex must be maintained throughout the period that the ds biomolecule is being translocated through the nanopore. If the complex is weak, or unstable, the complex can fall apart and will not be detected as the ds biomolecules thread through the pore. The stability is particularly important when the specific sequences to which the probe hybridize to are very short, for example, ˜6-15 bp long. Further, if the size/cross-sectional surface area of the complex is too small, the electric current differential/contrast produced is the signal amplitude when the complex thread through the pore is too small compared to the background noise and will not be detected.

Since the present invention uses PNA in order to increase the contrast in the change between the probe-biomolecules complex and other nucleic acid present in the sample, various strategies can be used to achieve that goal. For example, modification can be included in the pseudopeptide backbone to change the overall charge of the PNA to increase the contrast. Selection of more charged amino acids instead of non-polar amino acids serves to increase the charge of the PNA oligomer. In addition, small particle, molecules, protein, or peptides can be conjugated to the pseudopeptide backbone to enhance the bulk or cross-sectional surface area of the dsDNA-PNA complex. Enhance bulk serves to enhance the signal amplitude contrast so that any electrical current differential resulting from the increase in bulk can be easily detected. Examples of small particle, molecules, protein, or peptides can be conjugated to the pseudopeptide backbone include but are not limited to nanometer-sized gold particles (e.g. 3 nm), quantum dots, polyethylene glycol (PEG), polyvinyl pyrrolidone, polyvinyl alcohol, polyamino acids, divinylether maleic anhydride, N-(2-Hydroxypropyl)-methacrylamide, dextran, dextran derivatives including dextran sulfate, polypropylene glycol, polyoxyethylated polyol, heparin, heparin fragments, polysaccharides, cellulose and trypsin inhibitor. Method of conjugation of molecules are well know in the art, e.g. in U.S. Pat. Nos. 5,180,816, 6,423,685, 6,706,252, 6,884,780, and 7,022,673, which are hereby incorporated by reference in their entirety. Examples of some conjugating agents include but are not limited to ethylenediaminetetraacetic acid (EDTA), diethylenetriaminopentaacetic acid (DTPA), ethyleneglycol-0,0′-bis(2-aminoethyl)-N,N,N′,N′-tetraacetic acid (EGTA), N,N′-bis(hydroxybenzyl)ethylenediamine-N,N′-diacetic acid (HBED), triethylenetetraminehexaacetic acid (TTHA), 1,4,7,10-tetra-azacyclododecane-N,N′,N″,N′″-tetraacetic acid (DOTA), 1,4,7,10-tetraazacyclotridecane-1,4,7,10-tetraacetic acid (TITRA), 1,4,8,11-tetraazacyclotetradecane-N,N′,N″,N′″-tetraacetic acid (TETA), and 1,4,8,11-tetraazacyclotetradecane (TETRA).

In one embodiment, the at least one probe is a RNA, DNA or modified forms thereof. In some embodiments, the RNA, DNA or modified forms thereof is single stranded. The entire probe need not hybridize to the ds biomolecules; it is sufficient that some percentage of the probe hybridizes to the ds biomolecule. In one embodiment, at least 50% of the probe hybridizes to the ds biomolecule. In another embodiment, at least 20% of the probe hybridizes to the ds biomolecule. In other embodiments, at least 5%, at least 10%, at least 15%, at least 25%, at least 30%, at least 35%, at least 40% or at least 45% of the probe hybridizes to the ds biomolecule. In some embodiments, a single probe is a hybrid of PNA, RNA, or DNA. In some embodiments, the hybridization portion of the probe is a hybrid of PNA, RNA, or DNA. In some embodiments, at least 50% of the hybridization portion of the probe is a hybrid of PNA, RNA, or DNA. In another embodiment, at least 50% of the hybridization portion of the probe is a PNA. For example, if the hybridization portion of the probe with the ds biomolecules is 4 bp, then at least 2 bp of this a PNA.

In one embodiment, the RNA, DNA modified forms thereof is less than 20 bp. In one embodiment, the single stranded RNA, DNA modified forms thereof is between 3-50 bp, including all the whole integers between 4-50 bp, e.g. 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 41, 42, 43, 44, 45, 46, 47, 48, 49, and 50 bp.

In one embodiment, the at least one probe hybridizes or complementary base pairs with at least 4 bp on the ds biomolecules. In other embodiments, the at least one probe hybridize or complementary base pair with at least 6 bp, at least 8 bp, at least 10 bp, at least 12 bp, at least 14 bp, at least 16 bp, at least 18 bp, or at least 20 bp, including all the whole integers between 4-20 bp on the ds biomolecules, e.g. 5, 6, 7, 8, 9, 10, 11, 12, 13, 14, 15, 16, 17, 18, 19, and 20 bp on the ds biomolecules.

In one embodiment, a specific region on the ds biomolecules that is targeted by the at least one probe is <8 bp. This targeted region represents the region that will complementary base pair with the probe. In one embodiment, a specific region on the ds biomolecule that is targeted by the at least one probe is between 4-20 bp, including all the whole integers between 4-20 bp, e.g. 5, 6, 7, 8, 9, 10, 11, 12, 13, 14, 15, 16, 17, 18, 19, and 20 bp.

In one embodiment, a specific region on the ds biomolecules that can be targeted by a PNA is comprised primarily of homopurines, i.e. adenine and guanine. In some embodiments, the target region of a ds biomolecules invaded by PNA is between six to twelve nucleotides long including all the whole integers between 6-12 nucleotides, e.g. 7, 8, 9, 10, and 11 nucleotides. Examples of PNA target sequences are GAAAGAAG (SEQ. ID. No. 1), AAGGAAAG (SEQ. ID. No. 2) and AAGAAGG (SEQ. ID. No. 3). In some embodiments, the PNAs are not labeled, i.e. not tagged with a chromophore, radioisotope, protein etc.

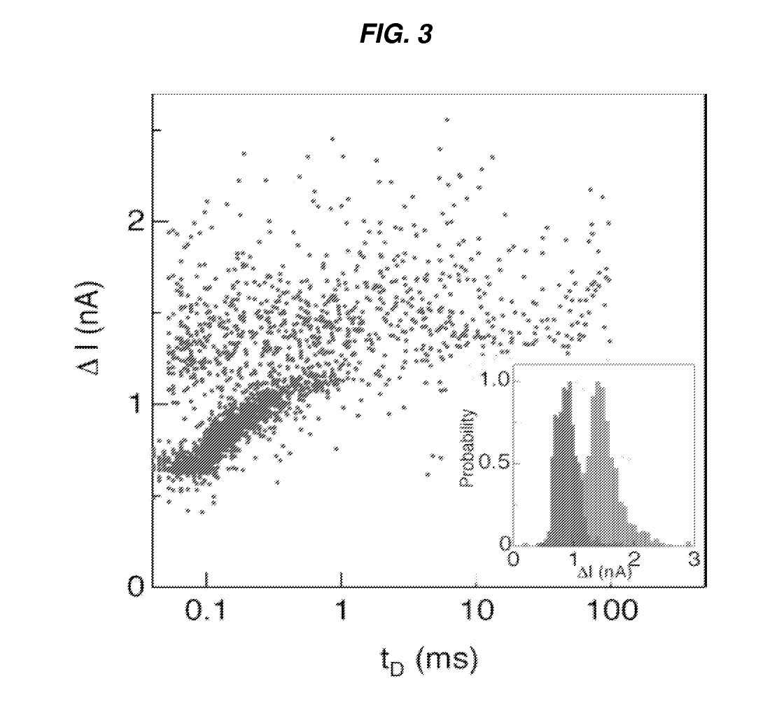

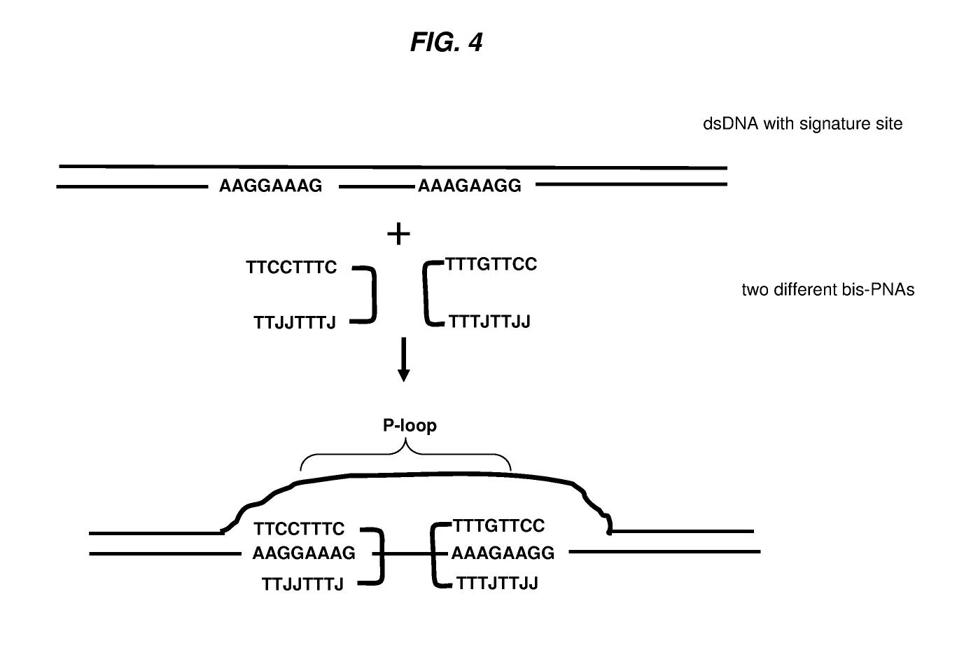

In one embodiment, a bis-PNA comprises two blocks of homopyrimidines (i.e. thymine and cytosine) connected by a flexible linker which can be purines, pyrimidines, or other modified or derivative forms of purines and pyrimidines. An example of a bis-PNA is TTCTTCCTGTJJTTJTT (SEQ. ID. No. 4). In some embodiments, the bis-PNAs are not labeled, i.e. not tagged with a chromophore, radioisotope, protein etc. BisPNAs are capable of targeting ds biomolecules in exceedingly sequence-specific manner via formation of a structure called P-loop (FIGS. 4 and 5), consisting of PNA₂-nucleic acid triplex with one of the two ds nucleic acid strands, while the other ds nucleic acid strand is displaced (Demidov, V. V. & Frank-Kamenetskii, M. D., Trends in Biochem. Sci. 29, 62-71 (2004); Egholm, M. et al., Nucleic Acids Res 23, 217-222 (1995); Demidov, V., Bukanov, N. O. & Frank-Kamenetskii, M. D., in Peptide nucleic acids: protocols and applications 266 (Horizen Scientific, Wymondham; 1999).

In one embodiment, contacting a ds biomolecules with PNA produces a ds biomolecule-PNA complex. In one embodiment, contacting a dsDNA with a bis-PNA produces a dsDNA-bis-PNA complex. In some embodiments, the ds biomolecule-PNA complex and/or dsDNA-PNA complex is not labeled.

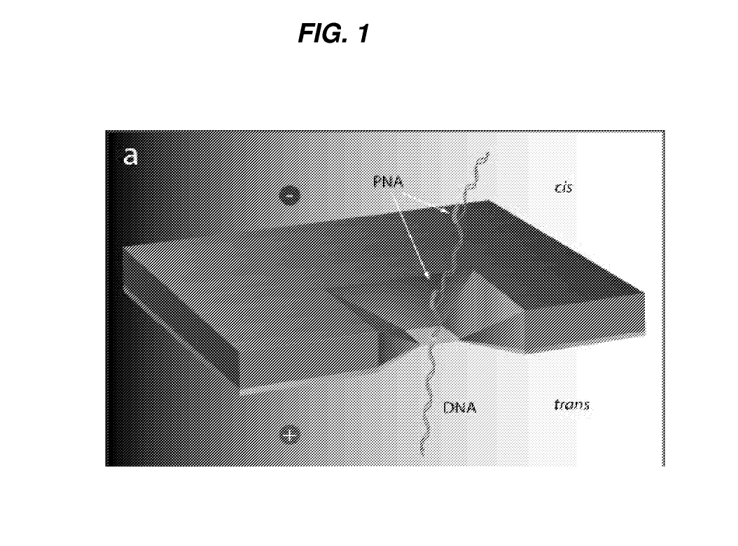

In one embodiment, the complexes formed described herein are triplexes, meaning that there are three strands of materials in the complex and these stand are complementary base pairing with each other (see FIG. 1c).

In one embodiment, the ds biomolecule-PNA complex or dsDNA-bis-PNA complex is detected by a micro-fluidic solid-state nanopore detection apparatus. The use of nanopore apparatus for the characterization of nucleic acid is well known in the art, see U.S. Pat. Nos. 5,795,782, 6,015,714, 6,362,002, and 6,428,959, and U.S. Patent Application Nos. 2003/0104428, 2007/0218471, 2007/0178507, and 2007/0048745. These references are incorporated herein by reference in their entirety. Ideally, the micro-fluidic solid-state nanopore detection apparatus comprises a first fluid chamber, a second fluid chamber, and a nanopore positioned between the first and second chambers such that the first and second chambers are in fluid communication via the nanopore. An electric potential is applied between the two chambers and the electric current flowing from the first chamber to the second chamber via the nanopore is monitored. Molecules that partially block the nanopore impede the electric current flowing through the nanopore and are registered as a drop in electric current flow with time. The drop in electric current flow with time varies proportionally with the size and configuration of the molecule partially blocking the nanopore. In the example described herein, the mixture sample comprising dsDNA F1 and probes P1 and P2, F1P1P2, registered an electric current change of 1.1 nA when just the duplex portion of the dsDNA was translocation through the nanopore. When the mixture sample comprising dsDNA F2 and probes P1 and P2, F2P1P2, translocated through the nanopore, two specific changes in the electric current change was noted and the change was larger at 1.5 nA. The electric current changes observed over time indicated the presences of distinct dsDNA-bis-PNA complexes at two specific regions on the dsDNA F2.

In one embodiment, the nanopore is between 3-6 nm in diameter. This size range is ideal for a single dsDNA molecule, with or without any dsDNA-PNA complex, to translocated through the nanopore. In one embodiment, the nanopore is 3.5 mm. In another embodiment, the nanopore is 4.5 nm. In another yet embodiment, the nanopore is 5 nm. In some embodiments, the nanopore size is up to 10 nm. All nanopore sizes between 3-10 nm are contemplated, e.g. 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9 and 10 nm.

In one embodiment, the electric potential across the first and second fluid chamber in the solid-state nanopore detection apparatus described herein is between 50-1000 mV. Generally, the electric potential applied for a nanopore of 5 nm is about 50-400 mV, more typically 100-200 mV.

In one embodiment, a plurality of probes is used. In other embodiments, at least two probes are used, at least three probes, at least four probes, at least five probes, at least six probes, at least seven probes, at least eight probes, at least nine probes, at least ten probes, at least 11 probes, at least 12 probes, at least 13 probes, at least 14 probes, at least 15 probes, at least 16 probes, at least 17 probes, at least 18 probes, at least 19 probes, at least 20 probes are used. Ideally, up to 20 different probes can be used on the ds biomolecule. Each probe has a unique sequence which hybridizes to a specific sequence on the ds biomolecule. In some embodiments, the probes attach or hybridize to different portions of the ds biomolecule. In some embodiments, the different portions of the ds biomolecule where the probes hybridize are at least 100 bp, at least 150 bp, at least 200 bp, at least 250 bp, at least 300 bp, at least 350 bp, at least 400 bp, at least 450 bp, at least 500 bp, at least 550 bp, at least 600 bp, at least 650 bp, at least 700 bp, at least 750 bp, at least 800 bp, at least 850 bp, at least 900 bp, at least 950 bp, or at least 1000 bp apart, including all the whole integers between 20-1000 bp.

For example, the plurality of probes can be designed to bind to specific polymorphisms or SNPs that one is looking for. In one embodiment, the plurality of probes can be designed for “barcoding” a biomolecule. Since the location/distributions of probes on a biomolecule is registered as changes in electric current over time, the pattern of these changes in electric current with time is unique for each plurality of probes used and serves as an identification pattern for the biomolecule having the specific sequences for the plurality of probes. In embodiment, DNA barcode uses a pattern of these changes in electric current with time to represent specific polymorphisms or SNPs. Barcoding is useful for distinguishing the stains and/or isolates of pathogens such as virus and bacteria.

The ability to detect single nucleic acid molecule of specific sequences can lead to cheaper and faster pathogen and mutation diagnostics platforms. Pathogens include bacteria, micro-organisms such as protists, viruses, including the various strains and isolates that have slight differences in the genome sequences. Accordingly, in one embodiment, provided herein is a method of detecting and diagnosing a drug resistant strain of pathogenic bacteria in a specimen under DNA non-denaturing conditions, the method comprising: providing a sample containing DNA; providing at least one probe having a known sequence that is unique to a drug resistant strain of pathogenic bacteria; contacting the at least one probe with the sample to produce a probe-DNA complex; introducing the probe-DNA complex into a microfluidic solid-state nanopore detection apparatus comprising a first fluid chamber, a second fluid chamber, a nanopore positioned between the first and second chambers such that the first and second chambers are in fluid communication via the nanopore; translocating the probe-DNA complex from the first chamber through the nanopore and into the second chamber by applying an electric potential between the two chambers; monitoring changes in electrical current across the nanopore as the probe-DNA complex is translocated therethrough and recording the changes in electrical current as a function of time, wherein the change in electrical current corresponding to presence of the probe-DNA complex containing the probe, indicating the presence of the drug resistant strain of pathogenic bacteria in the sample.

In one embodiment, provided herein is a method of detecting a drug resistant strain of Staphylococcus aureus in a specimen under DNA non-denaturing conditions, the method comprising: providing a sample containing DNA; providing at least one probe having a known sequence that is unique to a drug resistant strain of Staphylococcus aureus; contacting the at least one probe with the sample to produce a probe-DNA complex; introducing the probe-DNA complex into a microfluidic solid-state nanopore detection apparatus comprising a first fluid chamber, a second fluid chamber, a nanopore positioned between the first and second chambers such that the first and second chambers are in fluid communication via the nanopore; translocating the probe-DNA complex from the first chamber through the nanopore and into the second chamber by applying an electric potential between the two chambers; monitoring changes in electrical current across the nanopore as the probe-DNA complex is translocated therethrough and recording the changes in electrical current as a function of time, wherein the change in electrical current corresponding to presence of the probe-DNA complex containing the probe, indicating the presence of the drug resistant strain of Staphylococcus aureus in the sample.

In yet another embodiment, provided herein is a method of diagnosing a pathogenic bacteria or virus in a specimen under DNA non-denaturing conditions, the method comprising: providing a sample containing DNA; providing at least one probe having a known sequence that is unique to a pathogenic bacteria or virus; contacting the at least one probe with the sample to produce a probe-DNA complex; introducing the probe-DNA complex into a microfluidic solid-state nanopore detection apparatus comprising a first fluid chamber, a second fluid chamber, a nanopore positioned between the first and second chambers such that the first and second chambers are in fluid communication via the nanopore; translocating the probe-DNA complex from the first chamber through the nanopore and into the second chamber by applying an electric potential between the two chambers; monitoring changes in electrical current across the nanopore as the probe-DNA complex is translocated therethrough and recording the changes in electrical current as a function of time, wherein the change in electrical current corresponding to presence of the probe-DNA complex containing the probe, indicating the presence of the pathogenic bacteria or virus in the sample.

In humans it is estimated that on average 1 in 10³ nucleotides is variant between any two equivalent chromosomes. Although most such variations will be functionally neutral, a small proportion has been shown to underlie human phenotypic differences including the risk of disease. The present invention is applicable to these DNA variations, allowing them be investigated and detected. The DNA variations include but are not limited to single-nucleotide polymorphism (SNP) wherein the DNA sequence variation occurring when a single nucleotide in the genome differs between members of a species or between paired chromosomes in an individual, thus creating alleles. Variations in the DNA sequences of humans can affect how humans develop diseases and respond to pathogens, chemicals, drugs, vaccines, and other agents. For example, a SNP in the F5 gene causes a hypercoagulability disorder with the variant Factor V Leiden, single nucleic acid polymorphisms of OAS1 and MxA genes are associated with susceptibility to SARs, specific mutations on the BRAC1 and BRAC2 gene results in increased risk of breast cancer and increasing number of (CAG)ₙ repeats in Huntington's disease. There are known allelic mutations in the presenilin 1 gene (PSEN1), presenilin 2 gene (PSEN2), and the amyloid beta A4 precursor protein (APP) that cause early-onset Alzheimer's. The increase number of copies of the apolipoprotein E type 4 (Apoa4) variant allele is associated with the greater chance of developing Alzheimer disease Online Mendelian Inheritance in Man (OMIM) database at the United States National Library of Medicine has known SNPs and the related diseases and disorders.

According, in another embodiment, provided herein is a method of detecting and DNA variations such as SNPs, point mutations, polymorphisms, and polymorphic DNA biomarkers under DNA non-denaturing conditions, the method comprising: providing a sample containing DNA; providing at least one probe having a known sequence that is specific for the DNA variation; contacting the at least one probe with the sample to produce a probe-DNA complex, wherein the complex has sufficiently large cross section surface area that produces a detectable contrast in signal amplitude or electric current change over that of a background wherein the background in the signal amplitude or electric current corresponding to sections of non-PNA bound dsDNA.

In one embodiment, the methods described herein comprises introducing the probe-DNA complex into a microfluidic solid-state nanopore detection apparatus comprising a first fluid chamber, a second fluid chamber, a nanopore positioned between the first and second chambers such that the first and second chambers are in fluid communication via the nanopore; translocating the probe-DNA complex from the first chamber through the nanopore and into the second chamber by applying an electric potential between the two chambers; monitoring changes in electric current across the nanopore as the probe-DNA complex is translocated therethrough and recording the changes in electrical current as a function of time, wherein the change in electrical current corresponding to presence of the probe-DNA complex containing the probe, indicating the presence of the DNA variation in the sample.

In some embodiments, the DNA variations include but are not limited to RFLP (restriction fragment length polymorphism), VNTR (variable number of tandem repeat), STR (short tandem repeat or microsatellite), SNP (single-nucleotide polymorphism) and CNV (copy-number variation).

In one embodiment, for the methods described herein, the DNA in the sample is a dsDNA.

In one embodiment, for the methods described herein, the at least one probe is a PNA. In another embodiment, for the method described herein, the probe is a bis-PNA

In one embodiment, for the methods described herein, the at least one probe targets specific signature site on the dsDNA. These signature sites have sequences and/or sequence organizations that are unique to any desired biomarker of a drug-resistant bacteria and/or pathogen. The signature sites in the genome of drug-resistant bacteria and/or pathogen comprise 6-12 homopurines which can be bound by PNA-based sequence specific binding discussed herein. The homopurine region constitutes the PNA-binding region of the signature site. In one embodiment, the signature site comprises 20-30 nucleotides. In another embodiment, the signature site comprises two homopurine PNA binding region at the ends of the site. For example, a typical signature site is about 20-30 nucleotides long with two homopurine PNA binding region at the flanking ends of the signature site. In one embodiment, the two homopurine PNA binding region in the signature site each comprise 6-12 homopurines and each are separated from the other by a mixture of purines and pyrimidines. Encompassed herein is a signature site that can be bound by two different PNA at the two homopurine PNA binding regions (see FIG. 4). In some aspect, the PNAs bound at a signature site can be bis-PNA. As a result of the presence of the invasion and binding of the PNA or bis-PNAs, the duplex DNA at the signature site opens up, exposing one single DNA strand while the complementary DNA strand is paired with the PNA/bis-PNA in the formation of two dsDNA-PNA complexes in the dsDNA. This complex is stable. In one embodiment, the complexes are triplets comprising three strands of materials, e.g. DNA or PNA.

In one embodiment, for the methods described herein, the specific signature site on the dsDNA is bound by a pair of probes. In one embodiment, the pair of probes for the specific signature site is at least 4 bp apart and up to 20 bp apart, including all the whole integers between 4-20 bp.

In one embodiment, for the methods described herein, the complexes formed described herein are triplexes, meaning that there are three strands of materials in the complex and these stand are complementary base pairing with each other (see FIG. 4).

In one embodiment, for any of the methods described herein, the nanopore size ranges is between 3-6 nm in diameter. This size range is ideal for a single dsDNA molecule, with or without any dsDNA-PNA complex, to translocate through the nanopore. In one embodiment, the nanopore is 3.5 mm. In another embodiment, the nanopore is 4.5 nm. In another embodiment, the nanopore is 5 nm. In some aspects, the size of the nanopore can be up to 10 nm, especially when the probe has other molecules or particle conjugated to it to increase cross-sectional surface area for enhancing signal amplitude and signal contrast.

In one embodiment, for the methods described herein, a plurality of probes is used. In other embodiments, at least two probes are used, at least three probes, at least four probes, at least five probes, at least six probes, at least seven probes, at least eight probes, at least nine probes, at least ten probes, at least 11 probes, at least 12 probes, at least 13 probes, at least 14 probes, at least 15 probes, at least 16 probes, at least 17 probes, at least 18 probes, at least 19 probes, at least 20 probes are used. Ideally, up to 20 different probes can be used on the dsDNA. Each probe has a unique sequence which hybridizes to a specific sequence on the dsDNA. In some embodiments, the probes attach or hybridize to different portions of the dsDNA.

In some embodiments, the different portions of the dsDNA where the probes hybridize are at least 50 bp, at least 100 bp, at least 150 bp, at least 200 bp, at least 250 bp, at least 300 bp, at least 350 bp, at least 400 bp, at least 450 bp, at least 500 bp, at least 550 bp, at least 600 bp, at least 650 bp, at least 700 bp, at least 750 bp, at least 800 bp, at least 850 bp, at least 900 bp, at least 950 bp, or at least 1000 bp apart, including all the whole integer between 10-1000 bp.

In one embodiment, the specimen used in the methods disclosed herein is a clinical specimen. The specimen comprises a mixture of pathogenic microorganisms such as bacteria, virus, or parasite cells and non-pathogenic microorganism cells such as human or mammalian cells. In one embodiment, the specimen contains a mixture of different types of pathogenic microorganisms such as several different strains of bacteria. In another embodiment, the clinical specimen can be a tissue sample, for example, from an infected wound, or a tumor, tissue, or organ biopsy. As used herein, the term “clinical specimen” refers to materials harvested from a patient with an ailment/disease/disorder with the purpose of diagnosing the ailment/disease/disorder. In another embodiment, the specimen is obtained from the group consisting of: blood, sputum, feces, saliva, body tissue, peritoneal fluid, synovial fluid, urine, and cerebrospinal fluid. For example, the specimen is a blood sample drawn from an individual with chronic bacterial infection. This blood sample is used to for the detection of specific biomarker signature sites known to be found in certain drug-resistant bacteria. Positive detection of the biomarker signature sites in the blood sample indicates presence of the drug-resistant bacteria and the physician can now prescribe more potent medication that targets the drug-resistant bacteria.

In other embodiments, the methods described herein can be used to detect and identify polymorphisms, SNPs or biomarker signature sites that one is looking for in the drug-resistant bacteria/pathogen. In one embodiment, the methods described herein can be used to “barcoding” drug-resistant bacteria/pathogen.

In one embodiment, the specimen used in the methods disclosed herein is an environmental specimen. The specimen can comprise a mixture of DNA pathogenic microorganism. The specimen can be samples of soil, water, rain, sewage, air, food, dust, and solid surface wipes. Examples of pathogenic microorganisms in environmental samples include but are not limited to Salmonella sp., Streptococcus sp., Shigella sp., Botulism sp., Escherichia coli, Bacillus anthracia, Coliform bacteria, Vibrio cholrea, Giardia lamblia, and Hepatitis viruses A, B, and C.

The specimen is processed to separate the nucleic acid content from the protein content by any method known in the art, e.g. TRIZOL™ treatment or proteinase K treatment.

In one embodiment, the methods disclosed herein are used to diagnose clinically relevant pathogenic bacteria and the methods can diagnose up to three distinct bacteria in one specimen preparation simultaneously. By “clinically relevant pathogenic bacteria” is meant those bacteria that cause human and animal infections and diseases. Since the signature sites are unique to individual strains and species of bacteria, it is possible to simultaneously target three distinct signature sites representing the three different bacteria. This is performed using three distinct pairs of PNA probes that correspond to the selected three signature sites, one pair of PNA/signature site per bacteria of interest.

In one embodiment, the methods disclosed herein are used to diagnose clinically relevant pathogenic viruses and parasites that are associated with cancer and chronic disease, for example, Kaposi's sarcoma (gamma herpes viruses), Burkitt's lymphoma and Hodgkin's disease (Epstein Barr Virus and human T-lymphotropic virus type 1), cervical cancer (human pappiloma virus), liver cancer (hepatitis B and C viruses), and chronic cardiomyopathy on Chagas' disease (Trypanosoma cruzi).

In one embodiment, the clinically relevant pathogenic bacteria is selected from the group consisting of Clostridium botulism, Clostridium difficile, Bordetella pertussis, Listeria monocytogenes, Neisseria meningitides, Haemophilus influenzae, Brucella species, Coxiella burnetii, Shigella species, Escherichia coli O157:H7, Mycoplasma pneumoniae, Mycoplasma tuberculosis, Mycoplasma avium-intracellular complex, Mycoplasma gordonae, Mycoplasma kansaii, Staphylococci aurenus, Staphylococci epidermidis, Staphylococci saprophiticus, Staphylococci lugdunensis, Streptococcus pyogenes, Streptococcus agalactiae, Streptococcus pneumoniae, Enterococcus faecalis, Enterococcus faecium, Enterococcus gallinarum, Enterococcus casseliflavus, Klebsiella pneumoniae, Klebsiella oxytoca, Pseudomonas aeruginosa, Acinetobacter baumannii, Nocardia species, Salmonella species, Vibrio species, and Yersinia. Most of these pathogens are major causes of nosocomial and community-acquired infections, particularly in hospitals and healthcare setting.

In another embodiment, the methods disclosed herein are used to diagnose and distinguish drug-resistant strains of pathogens from non drug-resistant strains. There are increasing number of drug resistant strains of Staphylococci aurenus, Mycoplasma pneumoniae, Mycoplasma tuberculosis, Enterococcus species, Streptococcus pneumoniae, and Acinetobacter baumannii to name a few. These examples should not be construed to limiting as more drug-resistant strains of common bacteria are being isolated. The groups of drugs to which bacteria have acquired resistance are methicillin, macrolide, lincosamide, streptogamin, and vancomycin.

In another embodiment, the methods disclosed herein are used to detect, diagnose and distinguish toxin-producing strains of pathogens from non-toxin-producing strains of bacteria such as Clostridium botulism and Clostridium difficile. Rapid identification of toxin-producing strains of pathogens in an infection allows timely administration of drugs to counteract the effects of the bacterial toxins and buy the patient precious time for the antibiotics to neutralize the bacteria.

### PNA Design and Selection of Pathogenic Microorganism Signature Sites

Proper PNA design is crucial to the methods described herein. The optimal PNA design for sequence specific targeting of dsDNA is as follows. Unique constructs of PNA oligomers, called bisPNAs, which consist of two homopyrimidine PNA sequences connected with a flexible linker (FIG. 7a). Linkage of two PNA oligomers offers the potential to expediently design one PNA strand preferentially for Hoogsteen pairing and the other PNA strand preferentially for Watson-Crick pairing. This design approach is also advantageous in that it reduces a trimolecular reaction of PNA to DNA binding to a bimolecular reaction. It therefore results in enhanced PNA binding compared to monomeric PNAs. Optimal bisPNAs consist of normal pyrimidines (T and C nucleobases) in one oligomer sequence; in the other oligomer sequence all cytosines are replaced with pseudoisocytosines or J bases. The rational for such a substitution is that to form triplexes with the purine strand of the dsDNA during the P-loop assembly, Cs connected with Gs via Hoogsteen pairing must be protonated (see FIG. 7b) making the DNA/PNA complex formation highly pH-dependent. The use of J bases instead of Cs eliminates the pH dependence since no protonation is necessary (see FIG. 7c). In some exemplary bisPNAs shown herein, the PNA oligomers are flanked by three Lysine residues. Lysine carries a positive charge at a neutral pH making the inherently neutral PNA positively charged. This leads to two improvements: 1) increased PNA solubility and 2) superior PNA properties with regard to complex stability and dsDNA binding kinetics to dsDNA.

Numerous studies have shown that such bisPNA designs are highly efficient with respect to its exceedingly high sequence specific binding to bisPNA. The sequence discrimination of dsDNA targets by bisPNAs is kinetically controlled, so that both high complex stability and high specificity are achieved. BisPNAs with specific sequences for the project can be synthesized or purchase from Panagene Inc. which specializes in PNA synthesis.

The pair of PNA or bis-PNAs for each signature site is designed to have complementary sequences to the two flanking regions of a signature site; the flanking sites are thus called PNA-binding sites (see FIG. 4). These flanking regions or PNA binding sites contain approximately 6-12 nucleotide bases and are comprise of homopurines. The pair of PNA or bis-PNAs comprise homopyrimidines for complementary binding with the PNA-binding sites. Therefore, the pair of PNA or bis-PNAs are often called PNA-openers. The two selected PNA-openers will invade, bind, and hybridize to the same strand of DNA (the one with the homopurines PNA-binding sites) at the signature site and thus pry open the duplex DNA. This is the hallmark of the PNA technology.

In one embodiment, the signature target site can be located within a coding gene in an intron or a exon, or they can be found in the non-coding regions of the genome. The desirable features for a signature site are: (1) the sequence should be unique to that particular genus, species, strain, subtype, and isolate of pathogenic bacteria. For example, the signature target site should be used to distinguish between methicillin-sensitive and methicillin-resistant Staphylococcus aureus in a Staphylococcus infection, and also all mecA, nuc, tst, lukS-PV/lukF-PV and vanA/B strains. Similarly, the signature target site should also be able to distinguish Klebsiella pneumoniae from Pseudomonas aeruginosa in a blood infection. Other pathogens include cancer causing viruses such as gamma herpes viruses associated with Kaposi's sarcoma, Epstein-Barr virus (EBV) and Human T-lymphoma type 1 virus (HTLV-1) which predisposes infected individuals to cancers such as Burkitt's lymphoma and Hodgkin's disease, the human papilloma virus associated with cervical cancer, and hepatitis B and hepatitis C associated with liver cancer; (2) signature site is between 19-30 nucleotides long in the duplex DNA; (3) the signature site contains two homopurine sites at the flanking ends. The complementary strand of the signature site should have two homopyrimidine sites at the flanking ends. The common purines found in bacteria genome are adenine and guanine, and the complementary pyrimidines are thymine and cytosine; (4) the homopurine sites are between six to twelve nucleotides long; and (5) the two homopurine sites are separated by between two to twelve nucleotides which may be any combinations of purines and pyrimidines.

Using bacterial genome sequences available from Bacterial Genomes Database, signature sites with different distances and sequences between PNA-binding sites can be selected. The general formula used in the search of signature target sites is RₖNₙRₗ (where R is any purine and N is any base) choosing k and l between 6 and 12 and n between 2 and 12. On the basis of the formula and assuming that the sequence is random, a signature site is estimated to be found approximately every four hundreds base pairs. These signature target sites must be tested to select signature target sites that are unique for each chosen bacterial genome according to genomic BLAST program, which makes it possible to search for all signature target sites in all bacterial genomes with the given pattern.

A good number of signature sites are also identified using the method described herein for the pathogenic bacteria S. aureus and for distinguishing between the drug-resistant and non-drug resistant strains. Some examples of the number of signature sites identified by the method disclosed herein and examples of some signature site sequences of S. aureus are presented below, and should not be construed to be limited to only these examples.

The following is a list of signature sites chosen for their uniqueness in the respective bacteria's genome and are absent in genomes of the other sequenced bacteria. Additional exemplary bis-PNA tags and their locations on the genome of the pathogens are presented in Tables 1 and 2. This list should not be construed as limiting. The location of the signature sites in the genome are provided in parenthesis on the left. The bold and italic regions represent the homopurine flanking ends where the PNA-openers will bind, thus are also referred to as the PNA-binding sites.

Methicillin-Resistant Staphylococcus aureus (MRSA) (sites
identified in all SCC mec cassette types I-V and mec loci):
(SEQ. ID. No. 5) [symbol]
TTGTATTT [symbol]
(I, II, III, IV, V)
(SEQ. ID. No. 6) [symbol]
TAAATAC [symbol]
(I, II, III, IV, V)
(SEQ. ID. No. 7) [symbol]
TATTATTTCC [symbol]
(I, II, III, IV, V)
(SEQ. ID. No. 8) [symbol]
CAT [symbol]
(I, II, III, IV, V)
(SEQ. ID. No. 9) [symbol]
TATTGAT [symbol]
(I, II, III, IV, V)
(SEQ. ID. No. 10) [symbol]
CAT [symbol]
(I, II, III, IV, V)
(SEQ. ID. No. 11) [symbol]
TACAAATGC [symbol]
(II, III, mecR, mecI)
(SEQ. ID. No. 12) [symbol]
TTTATAGATCGT [symbol]
(II, III, mecR, mecI)
(SEQ. ID. No. 13) [symbol]
CAGAA [symbol]
(IV)
(SEQ. ID. No. 14) [symbol]
TACT [symbol]
(II, IV)
(SEQ. ID. No. 15) [symbol]
TCTTT [symbol]
(II, IV)
(SEQ. ID. No. 16) [symbol]
TGGAAGCACCT [symbol]
(I, II, IV)
(SEQ. ID. No. 17) [symbol]
TTAGAACAGCAC [symbol]
(I, II, IV)
(SEQ. ID. No. 18) [symbol]
CATGGGAT [symbol]
(I, II, IV)
Staphylococcus aureus toxic shock syndrome toxin-1 (tst) gene:
(SEQ. ID. No. 19) [symbol]
CTTACGATA [symbol]
(tst)
(SEQ. ID. No. 20) [symbol]
CT [symbol]
(tst)
Staphylococcus aureus Thermostable nuclease (nuc) gene:
(SEQ. ID. No. 21) [symbol]
CAATACGC [symbol]
(nuc)
(SEQ. ID. No. 22) [symbol]
TGT [symbol]
(nuc)
(SEQ. ID. No. 23) [symbol]
TGAAGCACAAGC [symbol]
(nuc)
Staphylococcus aureus at the lukS-PV, lukF-PV genes for
Panton-Valentine leukocidin gene:
(SEQ. ID. No. 24) [symbol]
CATAATTGATAT [symbol]
(SEQ. ID. No. 25) [symbol]
CTAATTTC [symbol]
(SEQ. ID. No. 26) [symbol]
CTC [symbol]
Vancomycin Resistance Staphylococcus aureus (VRSA),
on plasmid pLW043 (vanA):
(SEQ. ID. No. 27) [symbol]
CAATTACA [symbol]
(SEQ. ID. No. 28) [symbol]
TGGAT [symbol]
(SEQ. ID. No. 29) [symbol]
TTTCGTTCTC [symbol]
(SEQ. ID. No. 30) [symbol]
TCGAGCC [symbol]
(SEQ. ID. No. 31) [symbol]
CAGATTTACGC [symbol]
Clostridium botulism, in the bacterial genome
(SEQ. ID. No. 32) [symbol]
TGAGT [symbol]
(SEQ. ID. No. 33) [symbol]
ATATAG [symbol]
(SEQ. ID. No. 34) [symbol]
CAACT [symbol]
(SEQ. ID. No. 35) [symbol]
ACCTT [symbol]
(SEQ. ID. No. 36) [symbol]
TTAAGT [symbol]
(SEQ. ID. No. 37) [symbol]
ATC [symbol]
Clostridium botulism, in the plasmid pSOL1
(SEQ. ID. No. 38) [symbol]
CTGT [symbol]
Brucella species, in the bacterial genome
(SEQ. ID. No. 39) [symbol]
GCCGA [symbol]
(chromosome I)
(SEQ. ID. No. 40) [symbol]
GGC [symbol]
(chromosome I)
(SEQ. ID. No. 41) [symbol]
GCATTGC [symbol]
(chromosome I)
(SEQ. ID. No. 42) [symbol]
GAA [symbol]
(chromosome I)
(SEQ. ID. No. 43) [symbol]
CGGTCGCA [symbol]
(chromosome II)
(SEQ. ID. No. 44) [symbol]
TTGT [symbol]
(chromosome II)
Coxiella burnetii, in the bacterial genome
(SEQ. ID. No. 45) [symbol]
AAACTAC [symbol]
(SEQ. ID. No. 46) [symbol]
TCAGCTA [symbol]
(SEQ. ID. No. 47) [symbol]
AGCCGCTA [symbol]
(SEQ. ID. No. 48) [symbol]
CGTAA [symbol]
(SEQ. ID. No. 49) [symbol]
CGATCAT [symbol]
(SEQ. ID. No. 50) [symbol]
AATC [symbol]
(SEQ. ID. No. 51) [symbol]
GCTG [symbol]
Shigella species
(SEQ. ID. No. 52) [symbol]
TGTC [symbol]
(dysenteriae)
(SEQ. ID. No. 53) [symbol]
CTC [symbol]
(dysenteriae)
(SEQ. ID. No. 54) [symbol]
GGCGCGCC [symbol]
(flexneri)
(SEQ. ID. No. 55) [symbol]
TCCTTTG [symbol]
(boydii, sonnei)
(SEQ. ID. No. 56) [symbol]
ATTCACAG [symbol]
(Sb, Ss, Sd)
Escherichia coli, in the bacterial genome
(SEQ. ID. No. 57) [symbol]
AAG

[symbol]

In one embodiment, the methods of the invention do not comprise the signature target sequence of 5′-GAGGGAAGCCACGAGGGAGG_-3′ (SEQ. ID. No. 58) for HTVL-1 nor the signature target sequence of 5′-GGAAGAAGGCTAGGAAGAAG_-3′ (SEQ. ID. No. 59) for EBV.

In other embodiment, the methods of the invention described herein comprise the signature target sequences of 5′-GGAGAGAGACTCAAAAGAAGG-3′ (major cold-shock proteins) (SEQ. ID. No. 60), 5′-GAAAGAAGATGTGCTGAAAGAAG-3′ (RNA polymerase sigma N factor) (SEQ. ID. No. 61), 5′-GAAAGAAGAAGTGCCGGAAGAAG-3′ (Exoribonuclease R) (SEQ. ID. No. 62) for E. coli; 5′-GAAAAGAAACCCTTCAGAGGAAG-3′ (serA region) (SEQ. ID. No. 63), and 5′-GGAAGAAGCGCACTAAAGAAAA-3′ (yxjA gene) (SEQ. ID. No. 64) for B. subtilis; and 5′-AAAGAAAAATATTTAAAGAGGAA-3′ (dnaK region) (SEQ. ID. No. 65), 5′-AAAAGAGGTATTTTAAGAGGAA-3′ (wapA region) (SEQ. ID. No. 66), and 5′-GGAAGAAGTTCGGGTGAGAGGAAG-3′ (hypothetical protein region, hyp) (SEQ. ID. No. 67) for S. mutans.

Unless otherwise explained, all technical and scientific terms used herein have the same meaning as commonly understood by one of ordinary skill in the art to which this disclosure belongs. Definitions of common terms in molecular biology can be found in The Encyclopedia of Molecular Biology, published by Blackwell Science Ltd., 1994 (ISBN 0-632-02182-9); Robert A. Meyers (ed.), Molecular Biology and Biotechnology: a Comprehensive Desk Reference, published by VCH Publishers, Inc., 1995 (ISBN 1-56081-569-8); in Benjamin Lewin, Genes IX, published by Jones & Bartlett Publishing, 2007 (ISBN-13: 9780763740634); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN-1-56081-569-8).

Unless otherwise stated, the present invention was performed using standard procedures, as described, for example in Maniatis et al., Molecular Cloning: A Laboratory Manual, Cold Spring Harbor Laboratory Press, Cold Spring Harbor, N.Y., USA (1982); Sambrook et al., Molecular Cloning: A Laboratory Manual (2 ed.), Cold Spring Harbor Laboratory Press, Cold Spring Harbor, N.Y., USA (1989); Davis et al., Basic Methods in Molecular Biology, Elsevier Science Publishing, Inc., New York, USA (1986); Methods in Enzymology: Guide to Molecular Cloning Techniques Vol. 152, S. L. Berger and A. R. Kimmerl Eds., Academic Press Inc., San Diego, USA (1987)); Current Protocols in Molecular Biology (CPMB) (Fred M. Ausubel, et al. ed., John Wiley and Sons, Inc.) which are all incorporated by reference herein in their entireties.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used in connection with percentages may mean±1%.

The singular terms “a,” “an,” and the include plural referents unless context clearly indicates otherwise. Similarly, the word or is intended to include and unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The present invention can be defined in any of the following alphabetized paragraphs:
[A] A method for detecting a double-stranded (ds) biomolecule of interest comprising selecting at least one probe having a known sequence that hybridize by complementary base pairing to specific region on a ds biomolecule and contacting the at least one probe with the ds biomolecule such that the probe attaches to a specific region of the ds biomolecule to produce a probe-biomolecule complex, wherein the complex has sufficiently large cross-sectional surface area that produces a contrast in signal amplitude that is detectable.
[B] The method of paragraph [A], wherein the ds biomolecule is a dsDNA.
[C] The method of paragraph [A] or [B], wherein the at least one probe is a peptide-nucleic acid (PNA).
[D] The method of paragraph [C], wherein the PNA is a bis-PNA.
[E] The method of paragraph [D] wherein the probe-biomolecule complex is a triplex.
[F] The method of paragraph [C], wherein the PNA is a gamma-PNA.
[G] The method of paragraph [F], wherein the gamma PNA has at least one modified nucleobase, guanidinium G-clamp (X).
[H] The method of paragraph [F] or [G], wherein the gamma PNA has a higher binding affinity to the ds biomolecule.
[I] The method of paragraph [F], [G] or [H], wherein the gamma PNA forms five hydrogen bonds for at least one guanosine interaction with the ds biomolecule.
[J] The method of any paragraphs [A]-[I], wherein at least two probes are used, the probes attach to different specific regions of the ds biomolecule and the portions are at least 50 bp apart.
[K] 1The method of any paragraphs [A]-[J], wherein the detecting is by a microfluidic solid-state nanopore detection apparatus comprising a first fluid chamber, a second fluid chamber, a nanopore positioned between the first and second chambers such that the first and second chambers are in fluid communication via the nanopore, wherein an electric potential is applied between the two chambers and the electric current across the nanopore monitored.
[L] The method of paragraph [K], wherein the ds biomolecules and at least one probe are introduced to one of the chamber and the probe-biomolecule complex translocate through the nanopore in the presence of the electric potential is applied between the two chambers.
[M] The method of paragraph [K], wherein the nanopore is between 3-10 nm in diameter.
[N] The method of paragraph [K], wherein the electric potential in the solid-state nanopore detection apparatus is between 50-1000 mV.
[O] A method for detecting a double-stranded (ds) biomolecule of interest, the method comprising the steps of: providing a sample comprising a ds biomolecule; providing at least one probe having a known sequence; contacting the at least one probe with the ds biomolecule such that the probe hybridize by complementary base pairing to specific region on the ds biomolecule to produce a probe-biomolecule complex; introducing the probe-biomolecule complex into a microfluidic solid-state nanopore detection apparatus comprising a first fluid chamber, a second fluid chamber, a nanopore positioned between the first and second chambers such that the first and second chambers are in fluid communication via the nanopore; translocating the probe-biomolecule complex from the first chamber through the nanopore and into the second chamber by applying an electric potential between the two chambers; monitoring changes in current across the nanopore as the probe-biomolecule complex is translocated therethrough, the change in current corresponding to presence of the probe-biomolecule complex containing the probe; and recording the changes in electrical current as a function of time.
[P] The method of paragraph [O], wherein the ds biomolecule is a ds DNA.
[Q] The method of paragraph [O] or [P], wherein the at least one probe is a peptide-nucleic acid (PNA).
[R] The method of paragraph [Q], wherein the PNA is a bis-PNA.
[S] The method of paragraph [R], wherein the probe-biomolecule complex is a triplex.
[T] The method of paragraph [Q], wherein the PNA is a gamma-PNA.
[U] The method of paragraph [T], wherein the gamma PNA has at least one modified nucleobase, guanidinium G-clamp (X).
[V] The method of paragraph [T] or [U], wherein the gamma PNA has a higher binding affinity to the ds biomolecule.
[W] The method of paragraph [T], [U] or [V], wherein the gamma PNA forms five hydrogen bonds for at least one guanosine interaction with the ds biomolecule.
[X] The method of any of paragraphs [O]-[W], wherein the nanopore is between 3-10 nm in diameter.
[Y] The method of any of paragraphs [O]-[X], wherein the electric potential in the solid-state nanopore detection apparatus is between 50-1000 mV.
[Z] The method of any of paragraphs [O]-[Y], wherein at least two probes are used, the probes attach to different specific regions of the ds biomolecule and the portions are at least 100 bp apart.
[AA] A method of detecting and diagnosing a drug resistant strain of pathogenic bacteria in a specimen under DNA non-denaturing conditions, the method comprising: providing a sample containing DNA; providing at least one probe having a known sequence that is unique to a drug resistant strain of pathogenic bacteria; contacting the at least one probe with the sample to produce a probe-DNA complex; introducing the probe-DNA complex into a microfluidic solid-state nanopore detection apparatus comprising a first fluid chamber, a second fluid chamber, a nanopore positioned between the first and second chambers such that the first and second chambers are in fluid communication via the nanopore; translocating the probe-DNA complex from the first chamber through the nanopore and into the second chamber by applying an electric potential between the two chambers; monitoring changes in current across the nanopore as the probe-DNA complex is translocated therethrough and recording the changes in electrical current as a function of time, wherein the change in electrical current corresponding to presence of the probe-DNA complex containing the probe, indicating the presence of the drug resistant strain of pathogenic bacteria in the sample.
[BB] A method of detecting a drug resistant strain of Staphylococcus aureus in a specimen under DNA non-denaturing conditions, the method comprising: providing a sample containing DNA; providing at least one probe having a known sequence that is unique to a drug resistant strain of Staphylococcus aureus; contacting the at least one probe with the sample to produce a probe-DNA complex; introducing the probe-DNA complex into a microfluidic solid-state nanopore detection apparatus comprising a first fluid chamber, a second fluid chamber, a nanopore positioned between the first and second chambers such that the first and second chambers are in fluid communication via the nanopore; translocating the probe-DNA complex from the first chamber through the nanopore and into the second chamber by applying an electric potential between the two chambers; monitoring changes in current across the nanopore as the probe-DNA complex is translocating therethrough and recording the changes in electrical current as a function of time, wherein the change in electrical current corresponding to presence of the probe-DNA complex containing the probe, indicating the presence of the drug resistant strain of Staphylococcus aureus in the sample.
[CC] A method of diagnosing a pathogenic bacteria or virus in a specimen under DNA non-denaturing conditions, the method comprising: providing a sample containing DNA; providing at least one probe having a known sequence that is unique to a pathogenic bacteria or virus; contacting the at least one probe with the sample to produce a probe-DNA complex; introducing the probe-DNA complex into a microfluidic solid-state nanopore detection apparatus comprising a first fluid chamber, a second fluid chamber, a nanopore positioned between the first and second chambers such that the first and second chambers are in fluid communication via the nanopore; translocating the probe-DNA complex from the first chamber through the nanopore and into the second chamber by applying an electric potential between the two chambers; monitoring changes in current across the nanopore as the probe-DNA complex is translocated therethrough and recording the changes in electrical current as a function of time, wherein the change in electrical current corresponding to presence of the probe-DNA complex containing the probe, indicating the presence of the pathogenic bacteria or virus in the sample.
[DD] The method of any of paragraphs [AA]-[BB], wherein the DNA in the sample is a ds DNA.
[EE] The method of any of paragraphs [AA]-[BB], wherein the at least one probe is a PNA.
[FF] The method of paragraph [EE], wherein the PNA is a bis-PNA.
[GG] The method of paragraph [FF], wherein the probes-DNA complex is a triplex.
[HH] The method of paragraph [EE], wherein the PNA is a gamma-PNA.
[II] The method of paragraph [HH], wherein the gamma PNA has at least one modified nucleobase, guanidinium G-clamp (X).
[JJ] The method of paragraph [HH] or [II], wherein the gamma PNA has a higher binding affinity to the ds biomolecule.
[KK] The method of paragraph [HH], [II] or [JJ], wherein the gamma PNA forms five hydrogen bonds for at least one guanosine interaction with the ds biomolecule.
[LL] The method of any of paragraphs [AA]-[KK], wherein the nanopore is between 3-10 nm in diameter.
[MM] The method of any of paragraphs [AA]-[LL], wherein the electric potential in the solid-state nanopore detection apparatus is between 50-1000 mV.
[NN] The method of any of paragraphs [AA]-[MM], wherein at least two probes are used, the probes attach different portions of the ds DNA and the portions are at least 4 bp apart with in a signature site.
[OO] The method of any of paragraphs [AA]-[NN], wherein the specimen is a mixture of bacteria cells and non bacteria cells.
[PP] The method of any of paragraphs [AA]-[OO], wherein the specimen is a mixture of different types of bacteria.
[QQ] The method of any of paragraphs [AA]-[PP], wherein the specimen is obtained from the group consisting of: blood, sputum, feces, saliva, peritoneal fluid, synovial fluid, urine, body tissue, cerebrospinal fluid, soil, water, rain, sewage, air, food, dust, and solid surface wipes.
[RR] The method of paragraph [AA] or [CC], wherein the pathogenic bacteria is selected from the group consisting of Clostridium botulism, Clostridium difficile, Bordetella pertussis, Listeria monocytogenes, Neisseria meningitides, Haemophilus influenzae, Brucella species, Coxiella burnetii, Shigella species, Escherichia coli O157:H7, Mycoplasma pneumoniae, Mycoplasma tuberculosis, Mycoplasma avium-intracellular complex, Mycoplasma gordonae, Mycoplasma kansaii, Staphylococci aurenus, Staphylococci epidermidis, Staphylococci saprophiticus, Staphylococci lugdunensis, Streptococcus pyogenes, Streptococcus agalactiae, Streptococcus pneumoniae, Enterococcus faecalis, Enterococcus faecium, Enterococcus gallinarum, Enterococcus casseliflavus, Klebsiella pneumoniae, Klebsiella oxytoca, Pseudomonas aeruginosa, Acinetobacter baumannii, Nocardia species, Salmonella species, Vibrio species, and Yersinia.
[SS] The method of paragraphs [BB], wherein the drug resistant strain of Staphylococcus aureus is resistant to a group of drugs consisting of methicillin, clindamycin, ciprofloxacin and vancomycin.
[TT] The method of paragraph [AA], wherein the drug resistant strain pathogenic bacteria is resistant to a group of drugs consisting of methicillin, macrolide, lincosamide, streptogamin, and vancomycin.
[UU] The method of paragraph [AA], wherein the drug resistant strain pathogenic bacteria is selected from a group consisting of Staphylococcus, Steptococcus, Mycoplasma, Pneumococcus, Acinetobacter, and Entercoccous.

This invention is further illustrated by the following examples which should not be construed as limiting.

The contents of all references cited throughout this application, as well as the figures are incorporated herein by reference.

### Example 1

FIG. 1a shows a schematic of a nanopore system for the detection of PNA-DNA complexes in a ds DNA. A solitary 4-5 nm pore fabricated in a thin (˜20 nm) SiN membrane is assembled between two miniature fluid chambers (‘trans’ and ‘cis’), and hydrated using a 1M KCl buffered solution, as previously described (7). A voltage bias is then applied across the SiN membrane using a pair of AgCl electrodes. When the trans chamber is positively biased, DNA molecules in the cis chamber (4) thread through the nanopore into the trans chamber. To show that nanopores can discriminate among PNA-bound and PNA-free DNA samples, two DNA fragments (PCR-amplified from the -phage genome) of nearly equal lengths were have prepared (3,500 bp, see FIG. 1b). The first fragment (F1) serves as a negative control, which does not include target sequences for either of the two bis-PNA probes (PNA-1 and PNA-2). The positive sample (F2) contains two different binding sites for our bis-PNA probes (see sequences in FIG. 1c), spaced 855 bp apart. Binding of our probes to F2 was verified by a shift in mobility on a PAGE gel (see FIG. 1d). As seen from the Figure, the shift is greater when both probes are attached (Lane 4) than for either PNA-1 or PNA-2 (lanes 2 and 3, respectively). While the bis-PNA moieties marginally increase the molecular weights of the DNA, a more plausible explanation for the observed retarded mobility is the formation of a bend at the bis-PNA binding site (8-10). The occurrence of several bands in lanes 2-4 is due to the formation of structural isomers.

The nucleic acid complexes were further analyzed using the nanopore system. In FIG. 2 typical ion current traces are shown for the two samples, where both fragments were reacted with PNA-1 and PNA-2 prior to the nanopore experiment. FIGS. 2a and 2b show representative ion current traces of F1 and F2 (panel a and b respectively) translocation through a ˜4.5 nm pore, after incubation with the two PNA probes (P1 and P2). In the negative control experiment, characteristic signals from F1 show a single ‘blocked’ level reducing the ion current by roughly −1.1 nA which is in agreement with published results using similar length dsDNA. In contrast, signals from F2 molecules involve two distinct levels, at −1 nA and at −1.5 nA, as the all point histograms of the ion current suggest (see asterisks on left panels). All-point histograms of the two samples are shown to the right of each set of traces, clearly revealing the second characteristic event amplitude. The red curves are the time derivative of the current traces, used to automatically identify abrupt changes in current, attributed to the PNAs. These additional pulses in the experimental sample are much larger than the characteristic RMS noise in our signal (<0.02 nA @ 10 kHz low-pass filter), and therefore are attributed to the presence of the bis-PNA tags. Signals were digitally filtered at 1 KHz for display purpose. Moreover, machine-based identification of the additional current blockades can be implemented by analyzing the time derivative of the median-filtered raw data ('di/dt'), as shown by the red traces in FIG. 2. While entry/exit of DNA results in a large negative/positive spikes in the derivative, respectively, in the experimental sample additional sets of peaks were observed (the negative spikes are marked with arrows), corresponding to a local increase in excluded volume of the DNA induced by a bound bis-PNA moiety.

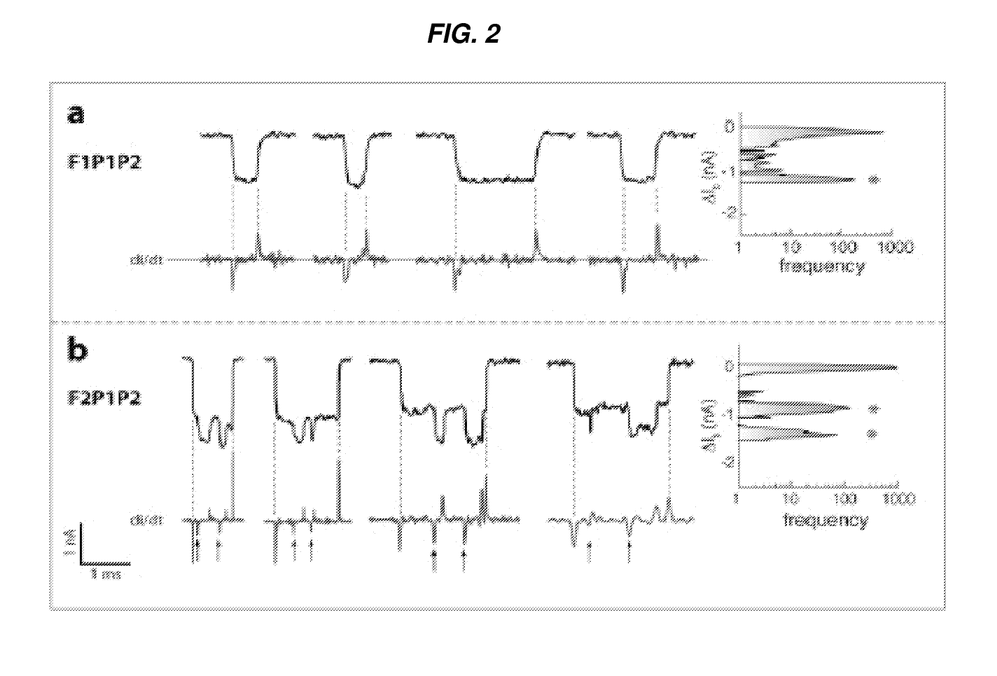

Surprisingly, the dwell times of the bis-PNA complexes in the pore were abnormally large, considering the fact that each complex spans only 8 bp, well under 1% of the DNA contour length. To address this, statistical analysis of ˜1,000 events in each of the control and experimental samples were conducted. FIG. 3 depicts scatter plots of the mean current amplitude (AI) vs. the total translocation time of the two samples. Two distinguishing features of the scatter plots are that the mean current amplitudes are larger and that the translocation times are longer (and more disperse) for the experimental sample. A more detailed analysis of the mean dwell-times of the bis-PNA complexes in the pore reveals that the DNA stalls at the point of the complex for a mean period of 0.1-1 ms, a major fraction of the total translocation time (see Supporting Information). Stalling at the complex site, while serendipitously enabling facile detection, presumably results from a combination of two factors a) increased interactions of the bulky complex with the pore, and b) formation of bends along the DNA molecule at the bis-PNA complex site, previously observed by electron microscopy and atomic force microscopy (8, 10).

In summary, the inventors have shown a novel single-molecule method for the tagging and subsequent identification of key sequences embedded within dsDNA. The ability to selectively tag DNA with spaced markers paves the way for DNA barcoding, involving more probes and variable spacing. This novel method does not require the use of fluorescence markers or dyes, and thus does not require the complex integration of optics. Yet due to its single-molecule nature an extremely high-sensitivity is obtained, even when using a few μl of sample. Furthermore, as relatively simple instrumentation is required, we hypothesize that the transformation of this technique into a broad ranging platform is in fact easily achievable. The potential to barcode DNA, and to read out key sequences without the need for amplification, labels or thermocycle processes as only miniscule DNA samples are required, opens up a broad range of possibilities in the fields of human genomics and pathogen detection in both the infectious and bio-terrorism fields.

### Example 2

In this example, the inventors show the use of two bis-PNA to create a P-loop and the detection of that P-loop. Two bisPNA probes were used to bind to two closely spaced binding sites on a linearized pUC19 plasmid. The two closely spaced binding sites represent the signature site in pathogen genome. As a result, an extended P-loop comprising of 19 nt in each strand was formed (FIG. 5). The target sequence was located approximately 500 bp from one end of the plasmid and 2200 bp from the other end, as shown in the figure. Complex formation was confirmed by nondenaturing gel electrophoresis, which showed a clear shift in the mobility of the DNA/PNA hybrid molecule as compared to the untagged DNA lane (data not shown).

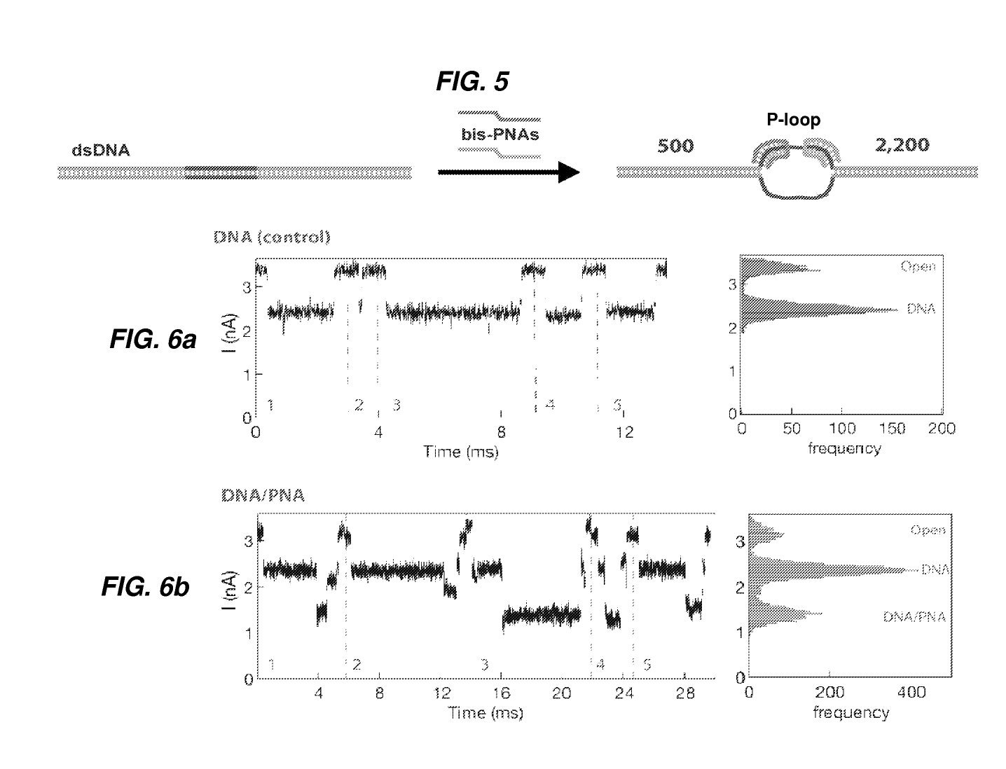

A 4 nm diameter nanopore was used to compare the hybridized complex with a control of intact, double-stranded linear pUC19. FIG. 6 shows a typical DNA translocation events (FIG. 6a) of the 2700 bp plasmid control DNA, and typical translocation events of the DNA/PNA complexes (FIG. 6b). The control DNA sample translocations exhibited a single blocked level at ˜2.4 nA and an open-pore level of ˜3.2 nA. In contrast, the DNA/PNA sample exhibited a distinctively different signal. A typical event began with a current drop from the open pore level to the characteristic dsDNA current level of ˜2.4 nA. The current then dropped a second time, to a lower blocking level of ˜1.4 nA. This lower current level is due to the presence of the bulky PNA tag in the pore. The current then returned to the dsDNA level and finally to the open pore current (see level histogram on the right hand side). The typical current level of the PNA/DNA tag's translocation is well below that of normal dsDNA, and resolving the two is well within the detection capabilities of our instrumentation.

Further data analysis of these preliminary nanopore experiments was performed to extract the dwell times of the dsDNA and DNA/PNA sections of the molecule in the pore. These dwell time distributions, constructed from hundreds of translocations similar to those shown in FIG. 5, allowed the inventors to measure the length of each individual section when compared to the work done previously in the PI's lab4. The shorter (500 bp) section (see FIG. 6) displays an average dwell time of 0.172 ms, while the longer (2200 bp) section takes an average of 1.75 ms to translocate through the pore. These dwell times are similar to those expected for dsDNA translocating through a nanopore under our experimental conditions. The tenfold difference in dwell times between the two sections, even with an un-optimized experimental system, indicates that the described detection method is sensitive to inter-tag distances on a fairly fine length scale. The dwell time for the PNA tagged section of the molecule is found to be surprisingly long, (0.458 ms) indicating that the PNA tag strongly interacts with the pore. This observation is also consistent with the fact that the PNA molecules are uncharged. The PNA tags increase the local cross section of the biopolymer, inducing stronger interactions with the pore, but the electrophoretic force is unchanged. Thus the net result is a slowing down of the translocation at the PNA-DNA location. The PNA-pore interaction, and hence the tag dwell time, is tunable through changes to the pore diameter and other experimental parameters, and should also be helpful in obtaining the maximum amount of information with which to characterize our molecule.

### Example 3

The nanopore based assays using γ-PNA probes are conducted in the same manner as for the nanopore based assays using bis-PNA except that the nanopore is 3.5 nm.

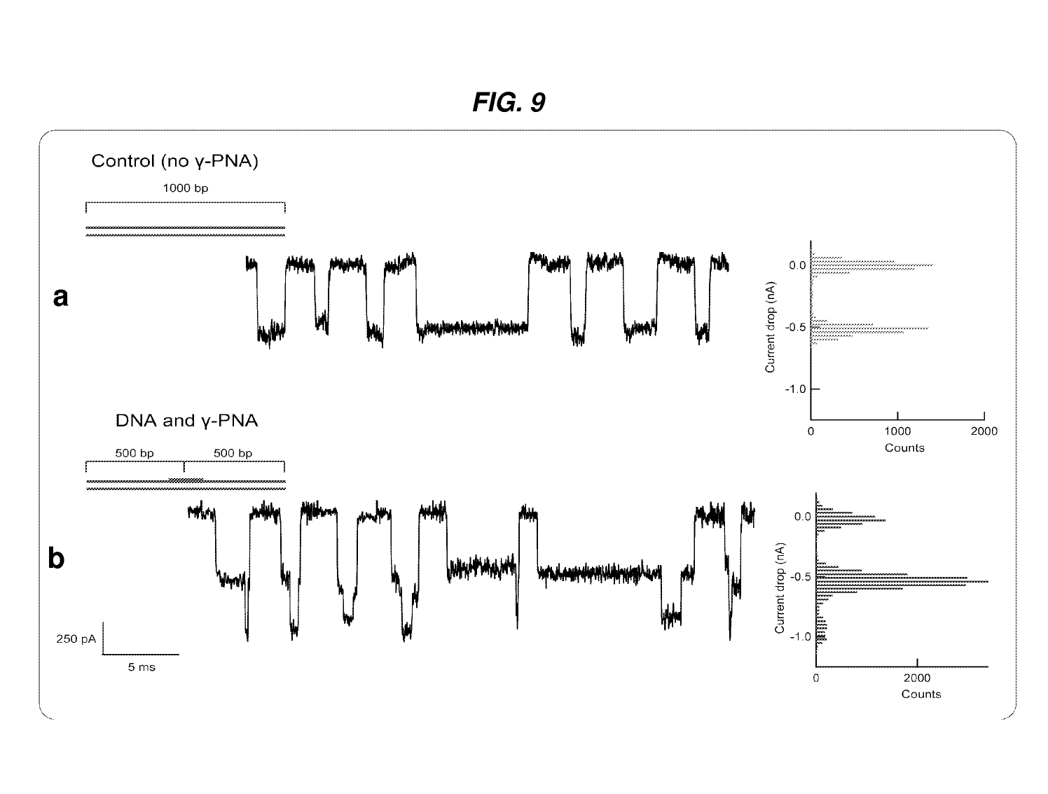

For this study, the inventors took a 1 kbp dsDNA molecule, with a single binding site located in the center (so ˜500 bp on either side of the γ-PNA binding site) and compared the readout signal from the nanopore based assay (FIG. 9). The parameters for the nanopore system are as described in Examples 1 and 2.

FIG. 9a shows an exemplary data obtained for the control experiment where no γ-PNA is added and mixed with the dsDNA, illustrating qualitatively an electrical current trace of a handful of single-molecule translocation events. Similar to FIGS. 2a and 6a in Examples 1 and 2 respectively, the single-molecule translocation events are seen as single block drop in the electrical current trace, which are identified quantitatively in an all-points histogram (FIG. 9a right side).

FIG. 9b shows an exemplary data obtained for the experiment where γ-PNA is added and mixed with the same 1 kbp dsDNA. The DNA was hybridized/invaded with the γ-PNA molecule and the hybridized/invadedregion on the dsDNA is in the center of 1 kbp dsDNA. As with FIG. 9a, a handful of single-molecule translocation events in an electrical current trace. Within each block drop in the electrical current trace representing a single-molecule translocation event, there is an additional drop in the electrical current trace. This additional drop represents the γ-PNA hybridized region in the dsDNA translocating through the nanopore. The presence of the γ-PNA causes a second deeper blockade of the nanopore which results in the additional drop in the electrical current trace while the dsDNA is translocating through the nanopore. The single-molecule translocation events are seen as single block drop in the electrical current trace, and the an additional drop in the electrical current trace representing the γ-PNA hybridized region in the dsDNA are identified quantitatively in an all-points histogram (FIG. 9b right side).

The references cited herein and throughout the specification are incorporated herein by reference.

### REFERENCES

1. Nielsen, P. E.; Egholm, M.; Berg, R. H.; Buchardt, O., Science 1991, 254 (5037), 1497-1500.
2. Pianowski, Z.; Gorska, K.; Oswald, L.; Merten, C. A.; Winssinger, N., Journal of the American Chemical Society 2009, 131 (18), 6492-97.
3. Phillips, K. M.; Larson, J. W.; Yantz, G. R.; D'Antoni, C. M.; Gallo, M. V.; Gillis, K. A.; Goncalves, N. M.; Neely, L. A.; Gullans, S. R.; Gilmanshin, R., Nucleic Acids Research 2005, 33 (18), 5829-5837.
4. Wanunu, M.; Sutin, J.; McNally, B.; Chow, A.; Meller, A., Biophys J 2008, 95 (10), 4716-25.
5. Wanunu, M.; Sutin, J.; Meller, A., Nano Lett 2009.
6. Smeets, R. M. M.; Kowalczyk, S. W.; Hall, A. R.; Dekker, N. H.; Dekker, C., Nano Letters 2009, Articles ASAP.
7. Wanunu, M.; Meller, A., Single-molecule techniques: a laboratory manual. Selvin, P. R.; Ha, T., Eds. Cold Spring Harbor Laboratory Press: Cold Spring Harbor, N.Y., 2008.
8. Bentin, T.; Nielsen, P. E., Nucleic Acids Research 2002, 30 (3), 803-809.
9. Crothers, D. M.; Drak, J.; Kahn, J. D.; Levene, S. D., Methods in Enzymology 1992, 212,3-29.
10. Chemy, D. I.; Fourcade, A.; Svinarchuk, F.; Nielsen, P. E.; Malvy, C.; Delain, E., Biophysical Journal 1998, 74 (2), 1015-1023.
11. Chema, V., S. Rapireddy, B. Sahu, C. Ausin, E. Pedroso, and D. H. Ly. Chem. BioChem 2008, 9:2388-2391.
12. Dragulescu-Andrasi, A., S. Rapireddy, B. M. Frezza, C. Gayathri, R. R. Gil, and D. H. Ly. J. American Chemical Society, 2006, 128:10258-10267.
13. He, G., S. Rapireddy, R. Bahal, B. Sahu, and D. H. Ly. J. American Chemical Society 2009, 131:12088-12090.
14. Rapireddy, S., G. He, S. Roy, B. A. Armitage, and D. H. Ly. J. American Chemical Society 2007, 129:15596-15600.

TABLE 1
Bis-PNA binding sites on RNA/DNA heteroduplexes obtained from RNA
bacteriophages Qbeta and MS2, for which model experiments will be performed

| | | RNA | Number of | |
|---|---|---|---|---|
| Phage | Accession^{a} | (nt) | binding sites^{b} | Exemplary bis-PNA tags (location)^{c} |
| Qbeta | NC_001890 | 4215 | 18 | N001 (951), N002 (1379), 1877 (2494), N003 |
| | | | | (3500), N004 (3839). |
| MS2 | NC_001417 | 3569 | 19 | N005 (388), 7308 (802), N006 (1802), N007 |
| | | | | (2022), 7260 (3054). |

^{a}NCBI reference sequences.
^{b}Sites for PNA tagging comprise octameric homopurine sequences. Sites may be located on the genomic RNA strand or on the synthesized DNA strand of the prepared RNA/DNA duplex.
^{b}Corresponding sequences are: PNA N001, GGAAGGAA (SEQ. ID. NO: 68); PNA N002, GGGAAGAA (SEQ. ID. NO: 69), PNA 1877, GGAAGAAG (SEQ. ID. NO: 70), PNA N003, GAGAAGAAG (SEQ. ID. NO: 71), PNA N004, AAGAAGAA (SEQ. ID. NO: 72), PNA N005, GAGGAGAA (SEQ. ID. NO: 73), PNA 7308, AAAGAAGG (SEQ. ID. NO: 74), PNA N006, GGAAGAAA (SEQ. ID. NO: 75), PNA N007, AGAGAAGG (SEQ. ID. NO: 75), PNA 7260, GGAGGAGA (SEQ. ID. NO: 77). Locations within the genomes denote the first nucleotide of a target sequence.

TABLE 2
Exemplary bis-PNA tags and locations
of their binding sites on NA of selected pathogens

| Pathogen | Accession^{a} | Size in nt | bis-PNA tags (location)^{b} |
|---|---|---|---|
| Australian bat | NC_003243 | 11822 | 7280 (107, 11465), 708 (637), 1126 (1035), 1877 |
| lyssavirus | | | (1398), 7231 (7067), 7308 (7666, 10424), 6798 |
| | | | (8436), 327 (11005), 1576 (11737) |
| Dengue | NC_001477 | 10735 | 1182 (1312, 2715), 1126 (1938), 1576 |
| | | | (2015, 2719, 8163), 708 (3032, 5074), 7280 |
| | | | (5894, 9365), 7260 (6241), 7308 (6272, 6853), |
| | | | 1877 (6368), 7231 (7709, 8836) |
| Hepatitis C virus | NC_004102 | 9646 | 1576 (364, 9465, 9505), 7260 (2726), 1182 |
| | | | (5087), 1126 (6554), 708 (9527, 5074), 7280 |
| | | | (5894) |
| Hepatitis E virus | NC_001434 | 7176 | 1126 (2243), 7231 (2498), 7308 (4541, 6726, |
| | | | 7124) |
| Parvovirus^{c} | NC_001358 | 5176 | 1576 (905), 1877 (1589), 7231 (2313), 327 (3088) |
| West Nile Virus | NC_009942 | 11029 | 1576 (402, 3860), 7231 (406), 7280 (2445), 7260 |
| | | | (3736, 10093), 1126 (7172, 9561, 10867), 7308 |
| | | | (7813, 9181, 9648), 6798 (7811) |

^{a}NCBI reference sequences.
^{b}DNA sites for PNA tagging comprise octameric homopurine sequences. Corresponding sequences are: PNA 327, AAGGGAAA (SEQ. ID. NO: 78); PNA 708, GAAAAGAA (SEQ. ID. NO: 79); PNA 1126, AGGGGAAG (SEQ. ID. NO: 80); PNA 1182, AAGGAAAG (SEQ. ID. NO: 81); PNA 1576, AAAGAAAA(SEQ. ID. NO: 82); PNA 1877, GGAAGAAG (SEQ. ID. NO: 83); PNA 6798, GAAAGAAG (SEQ. ID. NO: 1); PNA 7231, AAAAGAGG (SEQ. ID. NO: 84); PNA 7260, GGAGGAGA (SEQ. ID. NO: 77); PNA 7280, AGAGGAAG(SEQ. ID. NO: 85); PNA 7308, AAAGAAGG (SEQ. ID. NO: 74). Locations within the genomes denote the first nucleotide of a target sequence.
^{c}In contrast to all other viruses in the table, which carry ssRNA, parvovirus carries ssDNA as genetic material.

1. A method for detecting a double-stranded (ds) biomolecule of interest comprising selecting at least one probe having a known sequence that hybridize by complementary base pairing to a specific region on a ds biomolecule and contacting the at least one probe with the ds biomolecule such that the probe attaches to a specific region of the ds biomolecule to produce a probe-biomolecule complex, wherein the complex has sufficiently large cross-sectional surface area that produces a contrast in signal amplitude that is detectable.

2. The method of claim 1, wherein the ds biomolecule is a dsDNA.

3. The method of claim 1, wherein the at least one probe is a peptide-nucleic acid (PNA).

4. The method of claim 3, wherein the PNA is a bis-PNA or a gamma-PNA.

5. The method of claim 4, wherein the probe-biomolecule complex is a triplex.

6.-9. (canceled)

10. The method of claim 1, wherein at least two probes are used, the probes attach to different specific regions of the ds biomolecule and the portions are at least 50 bp apart.

11. The method of claim 1, wherein the detecting is by a microfluidic solid-state nanopore detection apparatus comprising a first fluid chamber, a second fluid chamber, a nanopore positioned between the first and second chambers such that the first and second chambers are in fluid communication via the nanopore, wherein an electric potential is applied between the two chambers and the electric current across the nanopore monitored.

12. The method of claim 11, wherein the ds biomolecules and at least one probe are introduced to one of the chamber and the probe-biomolecule complex translocate through the nanopore in the presence of the electric potential is applied between the two chambers.

13. The method of claim 11, wherein the nanopore is between 3-10 nm in diameter.

14. The method of claim 11, wherein the electric potential in the solid-state nanopore detection apparatus is between 50-1000 mV.

15. A method for detecting a double-stranded (ds) biomolecule of interest, the method comprising the steps of: providing a sample comprising a ds biomolecule; providing at least one probe having a known sequence; contacting the at least one probe with the ds biomolecule such that the probe hybridize by complementary base pairing to specific region on the ds biomolecule to produce a probe-biomolecule complex; introducing the probe-biomolecule complex into a microfluidic solid-state nanopore detection apparatus comprising a first fluid chamber, a second fluid chamber, a nanopore positioned between the first and second chambers such that the first and second chambers are in fluid communication via the nanopore; translocating the probe-biomolecule complex from the first chamber through the nanopore and into the second chamber by applying an electric potential between the two chambers; monitoring changes in current across the nanopore as the probe-biomolecule complex is translocated therethrough, the change in current corresponding to presence of the probe-biomolecule complex containing the probe; and recording the changes in electrical current as a function of time.

16. The method of claim 15, wherein the ds biomolecule is a ds DNA.

17. The method of claim 15, wherein the at least one probe is a peptide-nucleic acid (PNA).

18. The method of claim 17, wherein the PNA is a bis-PNA or a gamma-PNA.

19. The method of claim 18, wherein the probe-biomolecule complex is a triplex.

20.-25. (canceled)

26. The method of claim 15, wherein at least two probes are used, the probes attach to different specific regions of the ds biomolecule and the portions are at least 100 bp apart.

27. (canceled)

28. (canceled)

29. A method of detecting and diagnosing a pathogenic bacteria or virus in a specimen under DNA non-denaturing conditions, the method comprising: providing a sample containing DNA; providing at least one probe having a known sequence that is unique to a pathogenic bacteria or virus; contacting the at least one probe with the sample to produce a probe-DNA complex; introducing the probe-DNA complex into a microfluidic solid-state nanopore detection apparatus comprising a first fluid chamber, a second fluid chamber, a nanopore positioned between the first and second chambers such that the first and second chambers are in fluid communication via the nanopore; translocating the probe-DNA complex from the first chamber through the nanopore and into the second chamber by applying an electric potential between the two chambers; monitoring changes in current across the nanopore as the probe-DNA complex is translocated therethrough and recording the changes in electrical current as a function of time, wherein the change in electrical current corresponding to presence of the probe-DNA complex containing the probe, indicating the presence of the pathogenic bacteria or virus in the sample.

30. The method of claim 29, wherein the DNA in the sample is a ds DNA.

31. The method of claim 29, wherein the at least one probe is a PNA.

32. The method of claim 31, wherein the PNA is a bis-PNA or a gamma-PNA.

33. The method of claim 32, wherein the probes-DNA complex is a triplex.

34.-39. (canceled)

40. The method of claim 29, wherein at least two probes are used, the probes attach different portions of the ds DNA and the portions are at least 4 bp apart with in a signature site.

41.-42. (canceled)

43. The method of claim 29, wherein the specimen is obtained from the group consisting of: blood, sputum, feces, saliva, peritoneal fluid, synovial fluid, urine, body tissue, cerebrospinal fluid, soil, water, rain, sewage, air, food, dust, and solid surface wipes.

44. The method of claim 29, wherein the pathogenic bacteria is selected from the group consisting of Clostridium botulism, Clostridium difficile, Bordetella pertussis, Listeria monocytogenes, Neisseria meningitides, Haemophilus influenzae, Brucella species, Coxiella burnetii, Shigella species, Escherichia coli O157:H7, Mycoplasma pneumoniae, Mycoplasma tuberculosis, Mycoplasma avium-intracellular complex, Mycoplasma gordonae, Mycoplasma kansaii, Staphylococci aurenus, Staphylococci epidermidis, Staphylococci saprophiticus, Staphylococci lugdunensis, Streptococcus pyogenes, Streptococcus agalactiae, Streptococcus pneumoniae, Enterococcus faecalis, Enterococcus faecium, Enterococcus gallinarum, Enterococcus casseliflavus, Klebsiella pneumoniae, Klebsiella oxytoca, Pseudomonas aeruginosa, Acinetobacter baumannii, Nocardia species, Salmonella species, Vibrio species, and Yersinia.

45. (canceled)

46. The method of claim 29, wherein the pathogenic bacteria is a drug resistant strain pathogenic bacteria that is resistant to a group of drugs consisting of methicillin, macrolide, lincosamide, streptogamin, and vancomycin.

47. The method of claim 46, wherein the drug resistant strain pathogenic bacteria is selected from a group consisting of Staphylococcus, Steptococcus, Mycoplasma, Pneumococcus, Acinetobacter, and Entercoccous.


SEQUENCE LISTING (86 sequences)

SEQ ID NO: 1 - DNA, 8 bp, Artificial Sequence
  Description of Artificial Sequence Synthetic oligonucleotide
gaaagaag

SEQ ID NO: 2 - DNA, 8 bp, Artificial Sequence
  Description of Artificial Sequence Synthetic oligonucleotide
aaggaaag

SEQ ID NO: 3 - DNA, 7 bp, Artificial Sequence
  Description of Artificial Sequence Synthetic oligonucleotide
aagaagg

SEQ ID NO: 4 - DNA, 17 bp, Artificial Sequence
  Description of Artificial Sequence Synthetic oligonucleotide
ttcttcctgtccttctt

SEQ ID NO: 5 - DNA, 25 bp, Staphylococcus aureus
gaagaaattgtatttaagggagaag

SEQ ID NO: 6 - DNA, 26 bp, Staphylococcus aureus
agaagaaggtaaatacggagaagaag

SEQ ID NO: 7 - DNA, 28 bp, Staphylococcus aureus
ggaagaaaaatattatttccaaagaaaa

SEQ ID NO: 8 - DNA, 18 bp, Staphylococcus aureus
gaaaaaggcatgaaaaaa

SEQ ID NO: 9 - DNA, 23 bp, Staphylococcus aureus
aaggaggatattgatgaaaaaga

SEQ ID NO: 10 - DNA, 21 bp, Staphylococcus aureus
ggaagaaaaagcataaaaaag

SEQ ID NO: 11 - DNA, 24 bp, Staphylococcus aureus
agaagaaatacaaatgcaaaagga

SEQ ID NO: 12 - DNA, 29 bp, Staphylococcus aureus
aaaaagggatttatagatcgtaaaaaaga

SEQ ID NO: 13 - DNA, 25 bp, Staphylococcus aureus
gaaagaaaagcagaagggagggaaa

SEQ ID NO: 14 - DNA, 19 bp, Staphylococcus aureus
agaagaatactaaaggaag

SEQ ID NO: 15 - DNA, 19 bp, Staphylococcus aureus
ggaaaaatctttaggagaa

SEQ ID NO: 16 - DNA, 25 bp, Staphylococcus aureus
aaggaagtggaagcacctaaaggaa

SEQ ID NO: 17 - DNA, 26 bp, Staphylococcus aureus
gaagaaattagaacagcacaaaaaga

SEQ ID NO: 18 - DNA, 25 bp, Staphylococcus aureus
aagaaaacatgggatgaaaagaaag

SEQ ID NO: 19 - DNA, 21 bp, Staphylococcus aureus
aaagggcttacgataaaaaaa

SEQ ID NO: 20 - DNA, 18 bp, Staphylococcus aureus
aaaaagaactaaaaaaag

SEQ ID NO: 21 - DNA, 22 bp, Staphylococcus aureus
gaaagggcaatacgcaaagagg

SEQ ID NO: 22 - DNA, 18 bp, Staphylococcus aureus
aaaaaaggtgtagagaaa

SEQ ID NO: 23 - DNA, 33 bp, Staphylococcus aureus
aagaaaaagtgaagcacaagcgaaaaaagagaa

SEQ ID NO: 24 - DNA, 27 bp, Staphylococcus aureus
gaaaggacataattgatatgaaaaaaa

SEQ ID NO: 25 - DNA, 22 bp, Staphylococcus aureus
aaaagaactaatttcaaaaaaa

SEQ ID NO: 26 - DNA, 17 bp, Staphylococcus aureus
gaaaaaggctcaggaga

SEQ ID NO: 27 - DNA, 23 bp, Staphylococcus aureus
aagagaaaacaattacagaagaa

SEQ ID NO: 28 - DNA, 23 bp, Staphylococcus aureus
gaaggaaagtggatagaagaaaa

SEQ ID NO: 29 - DNA, 24 bp, Staphylococcus aureus
gaaaagaatttcgttctcgaggaa

SEQ ID NO: 30 - DNA, 22 bp, Staphylococcus aureus
aggaagtcgagccggaaaaagg

SEQ ID NO: 31 - DNA, 23 bp, Staphylococcus aureus
ggagggcagatttacgcggaaag

SEQ ID NO: 32 - DNA, 23 bp, Clostridium botulism
aaagaaaatgagtaaaagaagaa

SEQ ID NO: 33 - DNA, 23 bp, Clostridium botulism
aaaagaggatatagaaaaagagg

SEQ ID NO: 34 - DNA, 21 bp, Clostridium botulism
aaaagaggcaactggaggaga

SEQ ID NO: 35 - DNA, 21 bp, Clostridium botulism
aaggaaagaccttaaagaaaa

SEQ ID NO: 36 - DNA, 23 bp, Clostridium botulism
aaggaaagttaagtaaagaggaa

SEQ ID NO: 37 - DNA, 19 bp, Clostridium botulism
aaagaaaaatcaagggaaa

SEQ ID NO: 38 - DNA, 20 bp, Clostridium botulism
gaaaagaactgtgaaagaag

SEQ ID NO: 39 - DNA, 21 bp, Brucella sp.
aagaggaagccgaagaggaag

SEQ ID NO: 40 - DNA, 19 bp, Brucella sp.
agaggaaggccaaagaagg

SEQ ID NO: 41 - DNA, 23 bp, Brucella sp.
ggaagaaggcattgcggaagaag

SEQ ID NO: 42 - DNA, 19 bp, Brucella sp.
gaaaagaagaaggaagaag

SEQ ID NO: 43 - DNA, 24 bp, Brucella sp.
gaaaagaacggtcgcagaaaagaa

SEQ ID NO: 44 - DNA, 20 bp, Brucella sp.
aagaggaattgtaggggaag

SEQ ID NO: 45 - DNA, 23 bp, Coxiella burnetii
aagggaaaaaactacggagagag

SEQ ID NO: 46 - DNA, 23 bp, Coxiella burnetii
aaagaaggtcagctagaaaagaa

SEQ ID NO: 47 - DNA, 24 bp, Coxiella burnetii
aaagaaaaagccgctaaaagaaaa

SEQ ID NO: 48 - DNA, 21 bp, Coxiella burnetii
aagaggaacgtaaaaagaagg

SEQ ID NO: 49 - DNA, 23 bp, Coxiella burnetii
aaagaaaacgatcatgagggaag

SEQ ID NO: 50 - DNA, 20 bp, Coxiella burnetii
ggaagaagaatcgaaagaag

SEQ ID NO: 51 - DNA, 21 bp, Coxiella burnetii
aaagaaaagctgaaaagaagg

SEQ ID NO: 52 - DNA, 20 bp, Shigella sp.
gaaaagaatgtcaaggaaag

SEQ ID NO: 53 - DNA, 19 bp, Shigella sp.
aagggaaactcaaagaaga

SEQ ID NO: 54 - DNA, 24 bp, Shigella sp.
ggaagaagggcgcgccaaagagag

SEQ ID NO: 55 - DNA, 23 bp, Shigella sp.
aaggaaagtcctttgaaagaaaa

SEQ ID NO: 56 - DNA, 24 bp, Shigella sp.
aaagaaaaattcacagaaggaaag

SEQ ID NO: 57 - DNA, 19 bp, Escherichia coli
aagaggaaaagaaagaagg

SEQ ID NO: 58 - DNA, 20 bp, Human T-lymphotropic virus type 1
gagggaagccacgagggagg

SEQ ID NO: 59 - DNA, 20 bp, Epstein-Barr virus
ggaagaaggctaggaagaag

SEQ ID NO: 60 - DNA, 21 bp, Escherichia coli
ggagagagactcaaaagaagg

SEQ ID NO: 61 - DNA, 23 bp, Escherichia coli
gaaagaagatgtgctgaaagaag

SEQ ID NO: 62 - DNA, 23 bp, Escherichia coli
gaaagaagaagtgccggaagaag

SEQ ID NO: 63 - DNA, 23 bp, Bacillus subtilis
gaaaagaaacccttcagaggaag

SEQ ID NO: 64 - DNA, 22 bp, Bacillus subtilis
ggaagaagcgcactaaagaaaa

SEQ ID NO: 65 - DNA, 23 bp, Streptococcus mutans
aaagaaaaatatttaaagaggaa

SEQ ID NO: 66 - DNA, 22 bp, Streptococcus mutans
aaaagaggtattttaagaggaa

SEQ ID NO: 67 - DNA, 24 bp, Streptococcus mutans
ggaagaagttcgggtgagaggaag

SEQ ID NO: 68 - DNA, 8 bp, Artificial Sequence
  Description of Artificial Sequence Synthetic oligonucleotide
ggaaggaa

SEQ ID NO: 69 - DNA, 8 bp, Artificial Sequence
  Description of Artificial Sequence Synthetic oligonucleotide
gggaagaa

SEQ ID NO: 70 - DNA, 8 bp, Artificial Sequence
  Description of Artificial Sequence Synthetic oligonucleotide
ggaagaag

SEQ ID NO: 71 - DNA, 9 bp, Artificial Sequence
  Description of Artificial Sequence Synthetic oligonucleotide
gagaagaag

SEQ ID NO: 72 - DNA, 8 bp, Artificial Sequence
  Description of Artificial Sequence Synthetic oligonucleotide
aagaagaa

SEQ ID NO: 73 - DNA, 8 bp, Artificial Sequence
  Description of Artificial Sequence Synthetic oligonucleotide
gaggagaa

SEQ ID NO: 74 - DNA, 8 bp, Artificial Sequence
  Description of Artificial Sequence Synthetic oligonucleotide
aaagaagg

SEQ ID NO: 75 - DNA, 8 bp, Artificial Sequence
  Description of Artificial Sequence Synthetic oligonucleotide
ggaagaaa

SEQ ID NO: 76 - DNA, 8 bp, Artificial Sequence
  Description of Artificial Sequence Synthetic oligonucleotide
agagaagg

SEQ ID NO: 77 - DNA, 8 bp, Artificial Sequence
  Description of Artificial Sequence Synthetic oligonucleotide
ggaggaga

SEQ ID NO: 78 - DNA, 8 bp, Artificial Sequence
  Description of Artificial Sequence Synthetic oligonucleotide
aagggaaa

SEQ ID NO: 79 - DNA, 8 bp, Artificial Sequence
  Description of Artificial Sequence Synthetic oligonucleotide
gaaaagaa

SEQ ID NO: 80 - DNA, 8 bp, Artificial Sequence
  Description of Artificial Sequence Synthetic oligonucleotide
aggggaag

SEQ ID NO: 81 - DNA, 8 bp, Artificial Sequence
  Description of Artificial Sequence Synthetic oligonucleotide
aaggaaag

SEQ ID NO: 82 - DNA, 8 bp, Artificial Sequence
  Description of Artificial Sequence Synthetic oligonucleotide
aaagaaaa

SEQ ID NO: 83 - DNA, 8 bp, Artificial Sequence
  Description of Artificial Sequence Synthetic oligonucleotide
ggaagaag

SEQ ID NO: 84 - DNA, 8 bp, Artificial Sequence
  Description of Artificial Sequence Synthetic oligonucleotide
aaaagagg

SEQ ID NO: 85 - DNA, 8 bp, Artificial Sequence
  Description of Artificial Sequence Synthetic oligonucleotide
agaggaag

SEQ ID NO: 86 - DNA, 15 bp, Artificial Sequence
  Description of Artificial Sequence Synthetic peptide nucleic acid
tngtaaatgncgcnc